(19) United States
(12) Patent Application Publication
(10) Pub. No.: US 2025/0064918 A1
(43) Pub. Date: Feb. 27, 2025

(54) NIPAH HENIPAVIRUS VIRUS REPLICON PARTICLES AND THEIR USE

(71) Applicant: The U.S.A., as represented by the Secretary, Department of Health and Human Services, Bethesda, MD (US)

(72) Inventors: Stephen R. Welch, Atlanta, GA (US); Michael K. Lo, Decatur, GA (US); Markus H. Kainulainen, Atlanta, GA (US); Jessica R. Spengler, Athens, GA (US); Christina F. Spiropoulou, Atlanta, GA (US); Stuart T. Nichol, Atlanta, GA (US)

(73) Assignee: The U.S.A., as represented by the Secretary, Department of Health and Human Services, Bethesda, MD (US)

(21) Appl. No.: 18/726,235

(22) PCT Filed: Dec. 30, 2022
(86) PCT No.: PCT/US2022/082645

§ 371 Date: Jul. 2, 2024

Related U.S. Application Data
(60) Provisional application No. 63/296,435, filed on Jan. 4, 2022.

(51) Int. Cl. A61K 39/155; A61K 39/00; A61P 37/04; C12N 7/00

(52) U.S. Cl. CPC A61K 39/155; A61P 37/04; C12N 7/00; A61K 2039/5258; C12N 2760/18223; C12N 2760/18234; C12N 2760/18252; C12N 2760/18262; C12N 2760/18271

(57) ABSTRACT

Nipah henipavirus (NiV) virus replicon particles (VRPs) are disclosed herein. These VRPs can be used to induce an immune response to NiV or Hendra virus (HeV). In some embodiments, the NiV VRP include a recombinant NiV genome, wherein the recombinant NiV genome comprises a deletion in a nucleic acid sequence encoding the F protein such that functional mature F protein cannot be produced from the recombinant NiV genome; and a NiV envelope comprising F, G and M proteins of NiV. These VRP can infect human cells but cannot produce NiV particles from the infected human cells. Immunogenic compositions including the NiV VRP are also disclosed. In some embodiments, methods are disclosed for producing NiV VRP. The use of the disclosed NiV VRP to induce an immune response is also disclosed.

### CROSS REFERENCE TO RELATED APPLICATIONS

This claims the benefit of U.S. Provisional Application No. 63/296,435, filed Jan. 4, 2022, which is incorporated herein by reference.

### FIELD OF THE DISCLOSURE

This relates to immunogenic compositions, specifically to a Nipah henipavirus (NiV) virus replicon particle (VRP), methods for producing these VRP, and the use of these VRP.

### REFERENCE TO AN ELECTRONIC SEQUENCE LISTING

The electronic sequence listing (Sequences.xml; Size: 28,088 bytes; and Date of Creation: Dec. 30, 2022) is herein incorporated by reference in its entirety.

### BACKGROUND

NiV is an enveloped non-segmented negative-strand RNA virus of the family Paramyxoviridae. The natural NiV host are fruit bats of the Pteropodidae Family. NiV infection in humans has a range of clinical presentations, from asymptomatic infection to acute respiratory syndrome and fatal encephalitis. About a quarter of the human patients have seizures and about 60% become comatose and might need mechanical ventilation. NiV is a highly pathogenic viral zoonoses with a case fatality rate of 50-80%. The virus is transmitted to humans either through direct or indirect contact with infected animals (mainly fruit bats of Pteropus species) or via person-to-person transmission, commonly seen in families and care providers of NiV-infected individuals. NiV is also capable of causing disease in pigs and other domestic animals.

The NiV viral envelope contains several membrane proteins, including an envelope fusion protein (F), and an attachment protein (G). The NiV G protein is a Type II membrane protein that facilitates attachment of NiV to host cell membranes. The NiV F protein is a Type I membrane protein that binds to a host cell receptor and facilitates fusion of host and viral membranes. NiV F is a class I fusion protein initially expressed as a single polypeptide precursor, designated F₀. F₀ trimerizes in the endoplasmic reticulum and is processed by a cellular protease at a conserved site generating, F₁ and F₂ polypeptides. The F₂ polypeptide originates from the N-terminal portion of the F₀ precursor and links to the F₁ polypeptide via disulfide bonds. The F₁ polypeptide anchors the mature F protein in the membrane via a transmembrane domain, which is linked to a cytoplasmic tail. Three protomers of the F₂-F₁ heterodimer assemble to form a mature F protein, which adopts a metastable “prefusion” conformation that is triggered to undergo a conformational change that fuses the viral and target-cell membranes.

Although NiV is known to contribute to human illness and disease burden, a vaccine for this virus is not available.

### SUMMARY OF THE DISCLOSURE

NiV virus replicon particles (VRPs) are disclosed herein. These VRPs can be used to induce an immune response to NiV and/or Hendra virus (HeV).

In one embodiment, the NiV VRP includes a recombinant NiV genome, wherein the recombinant NiV genome comprises a deletion in a nucleic acid sequence encoding the F protein such that functional mature F protein cannot be produced from the recombinant NiV genome; and a NiV envelope comprising F, G and M proteins of Nipah virus. These VRP can infect human cells but cannot produce NiV particles from the infected human cells. Immunogenic compositions including the NiV VRP are also disclosed.

In some embodiments, methods are disclosed for producing NiV VRP. The methods include expressing NiV F protein from a host cell that is stably transfected with a nucleic acid molecule encoding the NiV F protein operably linked to a promoter; and contacting the host cell with the disclosed NiV VRP, The NiV VRPs produced by the host cell are then collected.

The use of the disclosed NiV VRP to induce an immune response is also disclosed. In some embodiments, methods for inducing an immune response in a subject include administering an immunogenic composition that includes an effective amount of the disclosed VRP and a pharmaceutically acceptable carrier to the subject.

The foregoing and other features and advantages of the invention will become more apparent from the following detailed description of several embodiments which proceeds with reference to the accompanying figures.

### BRIEF DESCRIPTION OF THE FIGURES

FIGS. 1A-1B. Vero cells expressing NiV codon optimized F protein (Fco). Microscopy immunofluorescent images of control (1A) and engineered (1B) Vero cells where nuclei are visualized with DAPI and NiV-Fco is stained with α-NiV HMAF.

FIGS. 2A-2C. Characterization of a NiV VRP wherein the gene encoding F is deleted (NiVΔF VRP). 2A. Growth kinetics comparison of NiVΔF VRP in Vero-NiVFco cells versus wildtype NiV-M in Vero cells. Cells infected at MOI 0.1 and clarified supernatants analyzed at 24, 48, 72, and 96 hours post infection. 2B. Microscopy and immunofluorescence (IFA) images of Vero-NiVFco and Vero cells 72 hours post infection with either NiVΔF VRP or wildtype NiV-M at MOI 0.1. 2C. NGS analysis showing consensus sequence of NiVΔF (bottom) aligned to parental wildtype NiV-M.

FIG. 3. Efficacy of NiVΔF VRP vaccination in hamster challenged with NiV strain Malaysia. Represented are the mean weights, mean cumulative clinical scores, and survival of Syrian hamsters (groups of 8-10) vaccinated with 10⁶ TCID₅₀ of NiVΔF VRP either 28, 14, 7, 3, or 3+1, days prior to challenge with NiV-M. Top row shows hamsters challenged with 106 NiV-M intranasally, and bottom row shows hamsters challenged with 104 NiV-M intraperitoneally.

FIGS. 4A-4F. Sequence of an exemplary NiV Malaysia genome sequence (SEQ ID NO: 4), including the nucleic acid sequence encoding F protein.

FIGS. 5A-5C. NiVΔF demonstrates a non-pathogenic, phenotype in three highly susceptible small animal model of NiV disease. Represented are Kaplan-Meier survival curves of animals inoculated with NiVΔF: (A) 5-7 week old Syrian hamsters were intranasally (IN) inoculated with either 10⁶ TCID₅₀ of NiVΔF, or one of 3 dilutions of wildtype NiV ranging from 10⁶ to 10³ TCID₅₀; (B) 2-3 days old suckling mice were intracranially (IC) inoculated with either 10⁵ TCID₅₀ of NiVΔF, or one of 7 dilutions of wildtype NiV ranging from 10⁶ to 10⁰ TCID₅₀; and (C) IFNAR^{-/-} mice were challenged IP with either NiVΔF (10⁶ TCID₅₀,), or NiV strain Malaysia (10⁶ TCID₅₀, red; or 10³ TCID₅₀,). Significance calculated by log-rank (Mantel-Cox test): ****, p=<0.0001; ***, p=<0.001; **, p=<0.01; *, p=<0.05.

FIGS. 6A-6G. NiVΔF demonstrates a non-spreading phenotype and absence of histopathology in highly susceptible Syrian hamster model of NiV disease. 5-7 week old Syrian hamsters were inoculated either IN or SC with 10⁶ TCID₅₀ of NiVΔF and euthanized either 1, 3, 7, 14, and 28 days post vaccination (n=10 per group). (A) vRNA tissue levels at each time point were determined by RT-qPCR. (B-G) 5-7 week old Syrian hamsters were inoculated IN with 10⁶ TCID₅₀ of either NiVΔF or NiV and euthanized 1, 3, and 7 days post vaccination (n=4 per group) unless humanely euthanized earlier due to clinical signs (NiV-infected hamsters only): (B,C) Tissue (lung and brain) and (D,E) mucosal swab samples (oral and rectal) were taken at euthanasia to determine both vRNA levels (RT-qPCR) and quantify infectious virus titers (TCID₅₀) in sample types where NiVΔF RNA were detected. Significance calculated by t-test: *, p=<0.05; **, p=<0.01. Lung tissues were formalin fixed and evaluated at 1, 3, and 7 dpi by (F) hematoxylin and eosin stain to characterize tissue pathology, and by (G) in-situ hybridization (ISH) to detect viral RNA. NiVΔF and mock-inoculated lungs showed no significant histopathologic changes at any timepoint, while NiV-inoculated lungs showed progressive inflammation, with epithelial syncytia compatible with paramyxoviral pneumonia. By ISH, NiVΔF RNA was detected at 1 dpv and decreased by 7 dpv. In contrast, NiV RNA was detected at relatively higher levels at 1 dpi and increased over time with severity of pneumonia.

FIGS. 7A-7E. Immune responses after single-dose subcutaneous or mucosal delivery of NiVΔF. 5-7 week old Syrian hamsters were inoculated either IN or SC with 10⁶ TCID₅₀ of NiVΔF and euthanized either 1, 3, 7, 14, and 28 days post vaccination (n=10 per group). (A) IgG antibody titers against NiV N and G were determined by ELISA at all time points post vaccination. (B) For IN vaccinated hamsters, IgA titers against NiV F, N, and G were determined by ELISA 28 days post vaccination. (C) Neutralizing antibody titers against NiV strain Malaysia were evaluated 1, 3, 7, 14, and 28 days post vaccination. (D) Antibody dependent complement deposition (ADCD) assay showing complement fixing activity of antibodies, and (E) Antibody dependent cellular phagocytosis (ADCP) assay depicting phagocytic activity of antibodies, determined at 7, 14, and 28 days post vaccination with NiVΔF. In all panels individual values are shown, with bars representing mean and standard deviation. Significance calculated by t-test: *, p=<0.05; **, p=<0.01; ***, p=<0.001.

### SEQUENCE LISTING

The nucleic and amino acid sequences listed are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.
SEQ ID NO: 1 is an exemplary amino acid sequence of a NiV F protein.
SEQ ID NO: 2 is an exemplary amino acid sequence of a NiV G protein.
SEQ ID NO: 3 is an exemplary amino acid sequence of a NiV M protein.
SEQ ID NO: 4 is an exemplary NiV Malaysia genome sequence, including the nucleic acid sequence encoding F protein.
SEQ ID NO: 5 is an exemplary codon-optimized nucleic acid molecule encoding the F protein.
SEQ ID NO: 6 is an exemplary amino acid sequence of a HeV G protein.

### DETAILED DESCRIPTION OF SEVERAL EMBODIMENTS

NiV-derived VRPs are disclosed herein. A production system is disclosed that includes: a) a NiV-derived VRP in which the fusion protein (F) gene has been deleted (NiVΔF); and b) a cell line engineered to constituently express NiV F protein which allow high-titers of the replicon particle, NiVΔF, VRP to be generated. Hamsters vaccinated intranasally (IN) with a single dose of NiVΔF VRP and subsequently challenged with wildtype NiV strain Malaysia 28. In some embodiments, administration of the disclosed VRP induce an immune response to more than one 5 strain of NiV, including, but not limited to strain Malaysia and/or strain Bangladesh. In some embodiments, administration of the disclosed VRP induce an immune response to another henipavirus, such as HeV. In some embodiments, treated subjects showed no clinical reaction and were protected against development of clinical signs, weight loss, and lethality. The NiV replicon system combines the benefits of a live vaccine (efficacy, immunogenicity, scalability) with the inherent safety benefits of single-cycle replication.

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. As used herein, the singular forms “a,” “an,” and “the,” refer to both the singular as well as plural, unless the context indicates otherwise. For example, the term “a VRP” includes single or plural VRPs and can be considered equivalent to the phrase “at least one VRP.” As used 20 herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. To facilitate review of the various embodiments, the following explanations of terms are provided:

Adjuvant: A vehicle used to enhance antigenicity. In some embodiments, an adjuvant includes a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I: C, or PolyICLC. (See, e.g., Singh (ed.) Vaccine Adjuvants and Delivery Systems. Wiley-Interscience, 2007).

Administration: The introduction of a composition into a subject by a chosen route. Administration can be local or systemic. For example, if the chosen route is intranasal, the composition (such as a composition including a disclosed VRP) is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of an amino acid in a polypeptide with one or more different amino acids. In the context of a protein sequence, an amino acid substitution is also referred to as a mutation.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant NiV Fectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with NiV infection. In still other embodiments, the control is a historical control or standard reference value or range of values (such as a previously tested control sample, such as a group of NiV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as a VRP, that is sufficient to elicit a desired response, such as an immune response in a subject. It is understood that to obtain a protective immune response can require multiple administrations of a disclosed VRP, and/or administration of a disclosed VRP as the “prime” in a prime boost protocol. Accordingly, an effective amount of a disclosed VRP can be the amount sufficient to elicit a priming immune response in a subject that can be subsequently boosted to elicit a protective immune response.

In one example, a desired response is to inhibit or reduce or prevent NiV infection. The NiV infection does not need to be completely eliminated or reduced or prevented for the method to be effective. For example, administration of an effective amount of the agent can decrease the NiV infection (for example, as measured by infection of cells, or by number or percentage of subjects infected by NiV) by a desired amount, for example by at least 50%, at least 60%, at least 70%, at least 80%, at least 90%, at least 95%, at least 98%, or even at least 100% (elimination or prevention of detectable NiV infection), as compared to a suitable control.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Hendra virus: An enveloped non-segmented negative-sense single-stranded RNA virus of the family Paramyxoviridae. The Hendra virus (HeV) genome is ˜18,000 nucleotides in length and includes 6 genes encoding 9 proteins, including the glycoproteins G, and F. Exemplary native HeV strain sequences are known to the person of ordinary skill in the art. Several models of human HeV infection are available, including model organisms infected with HeV, such as ferrets, mice, golden hamsters, and African Green Monkeys (see, e.g., Rockx, Pathogens and Disease 2014, doi: 10.1111/2049-632X.12149, which is incorporated by reference herein in its entirety).

The natural HeV host are fruit bats of the Pteropodidae Family. HeV infection in humans is associated with acute respiratory syndrome and fatal encephalitis. Although infection with HeV in humans is rare, the case fatality rate is high (˜57%). HeV is also capable of causing disease in other domestic animals such as horses with a mortality rate of ˜80%.

Heterologous: Originating from a different genetic source. A “heterologous protein” or “heterologous virus” is a protein or virus derived from a source other than a strain of NiV.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogen: A compound, composition, or substance (for example, a VRP) that can elicit an immune response in an animal, including compositions that are injected or absorbed into an animal. Administration of an immunogen to a subject can lead to protective immunity against a pathogen of interest.

Immunogenic composition: A composition comprising a disclosed VRP that induces a measurable CTL response against NiV, or induces a measurable B cell response (such as production of antibodies) against NiV, when administered to a subject. For in vivo use, the immunogenic composition will typically include the VRP in a pharmaceutically acceptable carrier and may also include other agents, such as an adjuvant.

Inhibiting or treating a disease: Inhibiting the full development of a disease or condition, for example, in a subject who is at risk for a disease such as NiV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Nipah henipavirus (NiV): An enveloped non-segmented negative-sense single-stranded RNA virus of the family Paramyxoviridae. The NiV genome is ˜18,000 nucleotides in length and includes 6 genes encoding 9 proteins, including the glycoproteins G, and F. Exemplary native NiV strain sequences are known to the person of ordinary skill in the art. Several models of human NiV infection are available, including model organisms infected with NiV, such as ferrets, mice, golden hamsters, guinea pigs, and African Green Monkeys (see, e.g., Geisbert et al., Curr. Top. Microbiol. Immunol., 359:153-77, 2012, which is incorporated by reference herein in its entirety).

The natural NiV host are fruit bats of the Pteropodidae Family. NiV infection in humans has a range of clinical presentations, from asymptomatic infection to acute respiratory syndrome and fatal encephalitis. NiV is also capable of causing disease in pigs and other domestic animals. In humans, NiV infection typically presents as fever, headache and drowsiness. Cough, abdominal pain, nausea, vomiting, weakness, problems with swallowing and blurred vision are relatively common. About a quarter of the human patients have seizures and about 60% become comatose and might need mechanical ventilation. In patients with severe disease, their conscious state may deteriorate and they may develop severe hypertension, fast heart rate, and very high temperature.

NiV fusion (F) protein: An envelope glycoprotein of NiV that facilitates fusion of viral and cellular membranes. In nature, the F protein from NiV is initially synthesized as a single polypeptide precursor approximately 550 amino acids in length, designated F₀. F₀ includes an N-terminal signal peptide that directs localization to the endoplasmic reticulum, where the signal peptide is proteolytically cleaved. The remaining F₀ residues oligomerize to form a trimer and may be proteolytically processed by a cellular protease to generate two disulfide-linked fragments, F₁ and F₂. In NiV F the cleavage site is located approximately between residues 109/110. The smaller of these fragments, F₂, originates from the N-terminal portion of the F₀ precursor (approximately residues 25-109). The larger of these fragments, F₁, includes the C-terminal portion of the F₀ precursor (approximately residues 110-550) including an extracellular/lumenal region (approximately residues 110-495), and a transmembrane and cytosolic regions (approximately residues 495-550). The extracellular portion of the NiV F protein is the NiV Fectodomain, which includes the F₂ protein and the F₁ ectodomain. The fusion peptide is located at the N-terminal segment of the F1 ectodomain, at approximately residues 110-122.

The NiV F protein exhibits remarkable sequence conservation within NiV strain. Unless context indicates otherwise, the numbering of NiV F amino acids is made with reference to SEQ ID NO: 1, shown below:

(SEQ ID NO: 1)
MVVILDKRCYCNLLILILMISECSVGILHYEKLSKIGLVKGVTRK
YKIKSNPLTKDIVIKMIPNVSNMSQCTGSVMENYKTRLNGILTPI
KGALEIYKNNTHDLVGDVRLAGVIMAGVAIGIATAAQITAGVALY
EAMKNADNINKLKSSIESTNEAVVKLQETAEKTVYVLTALQDYIN
TNLVPTIDKISCKQTELSLDLALSKYLSDLLFVFGPNLQDPVSNS
MTIQAISQAFGGNYETLLRTLGYATEDFDDLLESDSITGQIIYVD
LSSYYIIVRVYFPILTEIQQAYIQELLPVSFNNDNSEWISIVPNF
ILVRNTLISNIEIGFCLITKRSVICNQDYATPMINNMRECLTGST
EKCPRELVVSSHVPRFALSNGVLFANCISVTCQCQTTGRAISQSG
EQTLLMIDNTTCPTAVLGNVIISLGKYLGSVNYNSEGIAIGPPVF
TDKVDISSQISSMNQSLQQSKDYIKEAQRLLDTVNPSLISMLSMI
ILYVLSIASLCIGLITFISFIIVEKKRNTYSRLEDRRVRPTSSGD
LYYIGT.

An NiV F protein is also disclosed in NCBI Reference Sequence NP_112026.1, as available on Dec. 2, 2021, which is incorporated by reference herein. In vivo, during an infection, three NiV F protomers oligomerize in the mature F protein, which adopts a metastable prefusion conformation that is triggered to undergo a conformational change to a postfusion conformation upon contact with a target cell membrane. This conformational change exposes a hydrophobic sequence which is located at the N-terminus of the F₁ ectodomain, and which inserts into the host cell membrane and promotes fusion of the membrane of the virus, or an infected cell, with the target cell membrane.

NiV attachment glycoprotein (G): An NiV envelope glycoprotein that is a type II membrane protein and facilitates attachment of NiV to host cell membranes. The full-length G protein has an N-terminal cytoplasmic tail and transmembrane domain (CT and TM, approximately amino acids 1-176), and an ectodomain (approximately amino acids 177-602). An exemplary NiV G protein sequence from a Malaysian stain is provided below as SEQ ID NO: 2, and can also be found as NCBI Reference Sequence NP_112027.1, as available on Dec. 2, 2021, which is incorporated by reference herein). An exemplary amino acid sequence is:

(SEQ ID NO: 2)
MPAENKKVRFENTTSDKGKIPSKVIKSYYGTMDIKKINEGLLDSK
ILSAFNTVIALLGSIVIIVMNIMIIQNYTRSTDNQAVIKDALQGI
QQQIKGLADKIGTEIGPKVSLIDTSSTITIPANIGLLGSKISQST
ASINENVNEKCKFTLPPLKIHECNISCPNPLPFREYRPQTEGVSN
LVGLPNNICLQKTSNQILKPKLISYTLPVVGQSGTCITDPLLAMD
EGYFAYSHLERIGSCSRGVSKQRIIGVGEVLDRGDEVPSLFMTNV
WTPPNPNTVYHCSAVYNNEFYYVLCAVSTVGDPILNSTYWSGSLM
MTRLAVKPKSNGGGYNQHQLALRSIEKGRYDKVMPYGPSGIKQGD
TLYFPAVGFLVRTEFKYNDSNCPITKCQYSKPENCRLSMGIRPNS
HYILRSGLLKYNLSDGENPKVVFIEISDQRLSIGSPSKIYDSLGQ
PVFYQASFSWDTMIKFGDVLTVNPLVVNWRNNTVISRPGQSQCPR
FNTCPEICWEGVYNDAFLIDRINWISAGVELDSNQTAENPVFTVF
KDNEILYRAQLASEDTNAQKTITNCFLLKNKIWCISLVEIYDTGD
NVIRPKLFAVKIPEQCT

An exemplary NiV G protein sequence from NiV G from a Bangladesh stain is disclosed in GENBANK® Reference No. AAY43916.1, which is incorporated by reference herein. As used herein, NiV G residue positioning is made with reference to the sequence of the set forth as SEQ ID NO: 2.

NiV Matix (M) protein: A protein that functions in assembly and budding of progeny virions at the plasma membrane. Monomeric NiV-M has a calculated molecular weight of 39 kD, NiV-M likely to exist in the cell in vivo predominantly as homodimers (or higher order oligomers). See Watkinson and Lee, FEBS Let. 590:2494-2511, 2016, incorporated herein by reference for a description of the function of NiV M protein. An exemplary amino acid sequence for NiV M protein is:

(SEQ ID NO: 3)
MEPDIKSISSESMEGVSDFSPSSWEHGGYLDKVEPEIDENGSMIP
KYKIYTPGANERKYNNYMYLICYGFVEDVERTPETGKRKKIRTIA
AYPLGVGKSASHPQDLLEELCSLKVTVRRTAGSTEKIVFGSSGPL
NHLVPWKKVLTSGSIFNAVKVCRNVDQIQLDKHQALRIFFLSITK
LNDSGIYMIPRTMLEFRRNNAIAFNLLVYLKIDADLSKMGIQGSL
DKDGFKVASFMLHLGNFVRRAGKYYSVDYCRRKIDRMKLQFSLGS
IGGLSLHIKINGVISKRLFAQMGFQKNLCFSLMDINPWLNRLTWN
NSCEISRVAAVLQPSIPREFMIYDDVFIDNTGRILKG.

The NiV M protein from a Malaysian strain is disclosed in NCBI Reference Sequence NP_112025.1, as available on Dec. 2, 2021, which is incorporated by reference herein.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost vaccination: An immunotherapy including administration of a first immunogenic composition (the primer vaccine) followed by administration of a second immunogenic composition (the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant virus is one that includes a genome that includes a recombinant nucleic acid molecule.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals. In an example, a subject is a human. In a particular example, the subject is a newborn infant. In an additional example, a subject is selected that is in need of inhibiting of a NiV infection. For example, the subject is either uninfected and at risk of NiV infection or is infected in need of treatment.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Under conditions sufficient for: A phrase that is used to describe any environment that permits a desired activity.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides, or DNA derived from them, or a VRP. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response. In one specific, non-limiting example, a vaccine prevents and/or reduces the severity of the symptoms associated with NiV infection and/or decreases the viral load compared to a control.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs do not include a virus genome. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354:53073, 2012).

Virus Replicon Particle (VRP): Replication-competent but non-spreading virus particles. In the context of the present disclosure, a NiV VRP is a viral particle that contains a recombinant Nipah henipavirus genome, wherein the recombinant Nipah henipavirus genome comprises a deletion in at least the nucleic acid sequence encoding the F protein such that functional mature F protein cannot be produced from the recombinant Nipah henipavirus genome. The Nipah henipavirus genome can contain other mutations, such as, but not limit to, the nucleic acid sequence encoding the G and/or M proteins. The Nipah virus replicon particle also includes a Nipah henipavirus envelope comprising F, G and M proteins of Nipah virus. The VRP can infect human cells, but cannot produce Nipah henipavirus particles from the infected human cells. Thus, Nipah virus VRP are capable of a single round of infection, including expression of viral nucleic acids and de novo viral protein synthesis, but cannot form new particles (due to the lack of functional F protein) and therefore cannot spread to neighboring cells. VRPs are not naturally occurring.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

### NiV VRP

NiV VRP are a safe and rapidly efficacious immunogenic composition (such as a vaccine) for protection against NiV infection. In particular embodiments, the disclosed NiV VRP contain a genome with one or more mutations in the nucleic acid sequence encoding the F protein, such that functional mature F protein cannot be produced from the recombinant genome. In the absence of exogenous F protein, these VRP are unable to produce new particles from infected cells, preventing spread within the immunized host and eliminating the risk of vaccine-induced pathogenicity. The data disclosed herein demonstrate that NiV VRP immunization is both safe and efficacious against virulent NiV challenge in a relevant animal model. Immunization with non-replicating NiV VRP resulted in a significant increase survival following Nipah henipavirus challenge. The animals also developed a strong immune response. The disclosed VRP can infect human cells, but cannot produce Nipah henipavirus particles from the infected human cells in the absence of exogenous F protein.

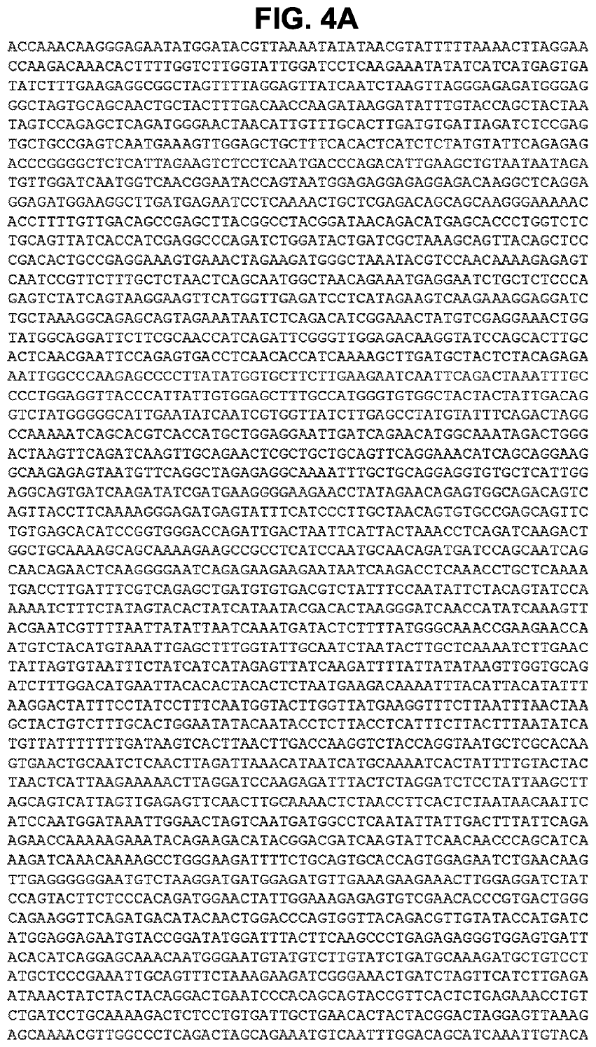

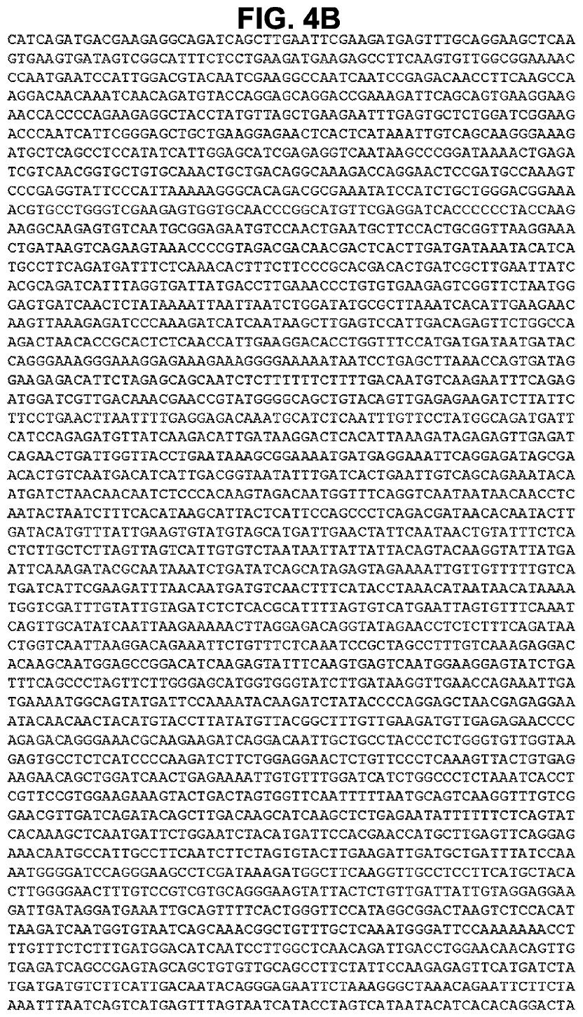

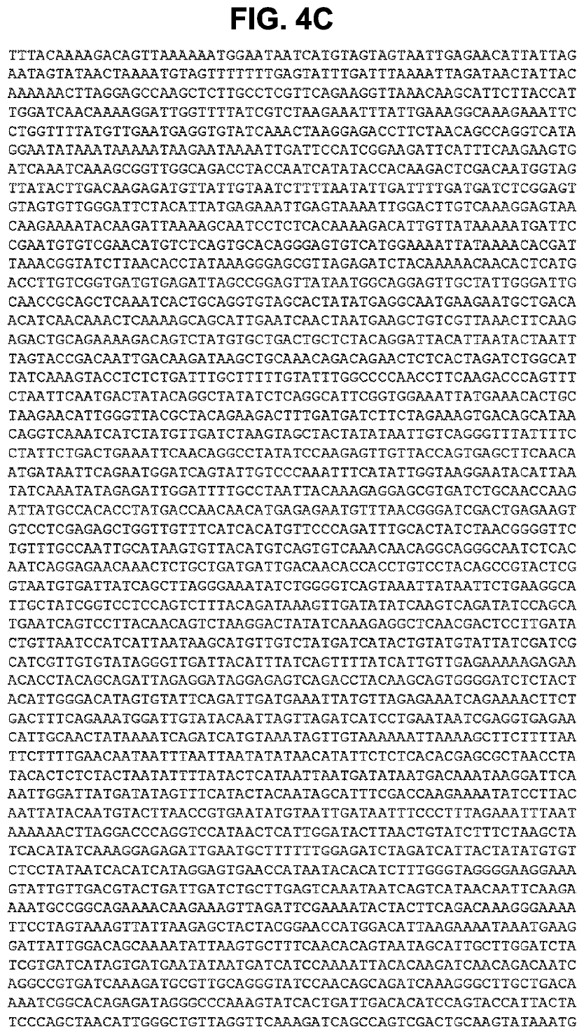

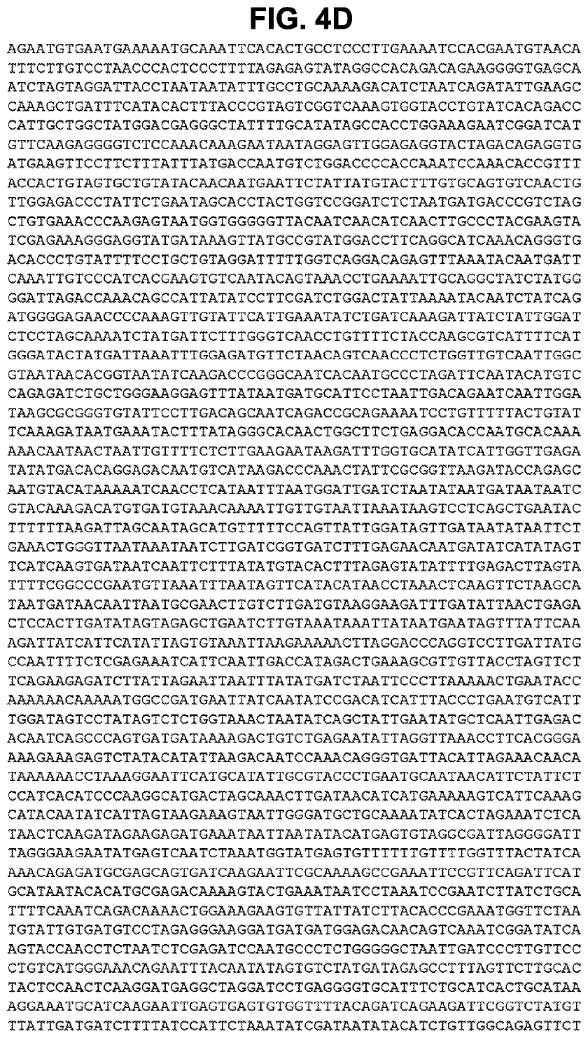

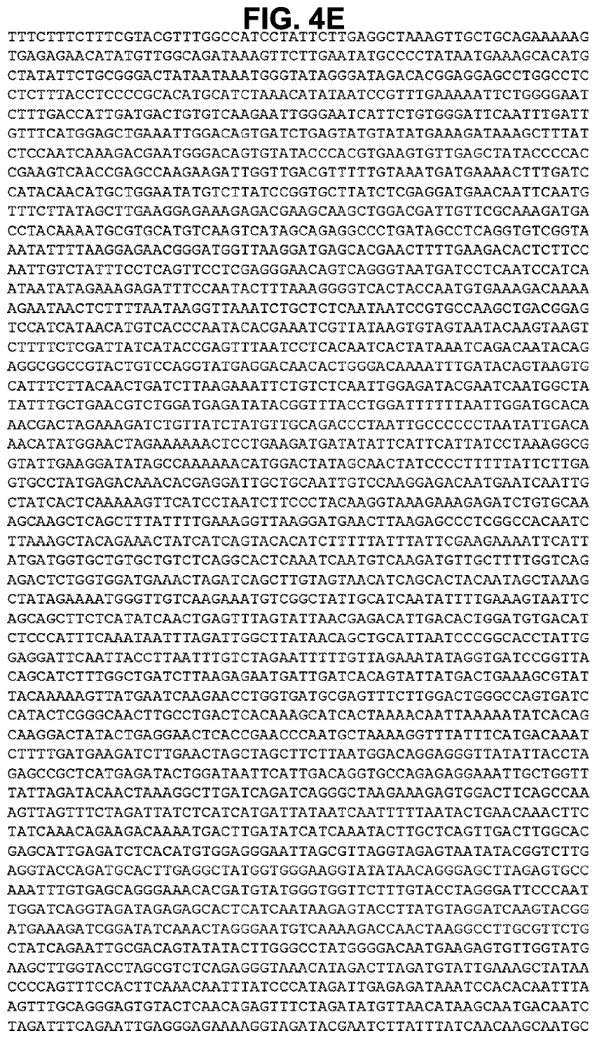

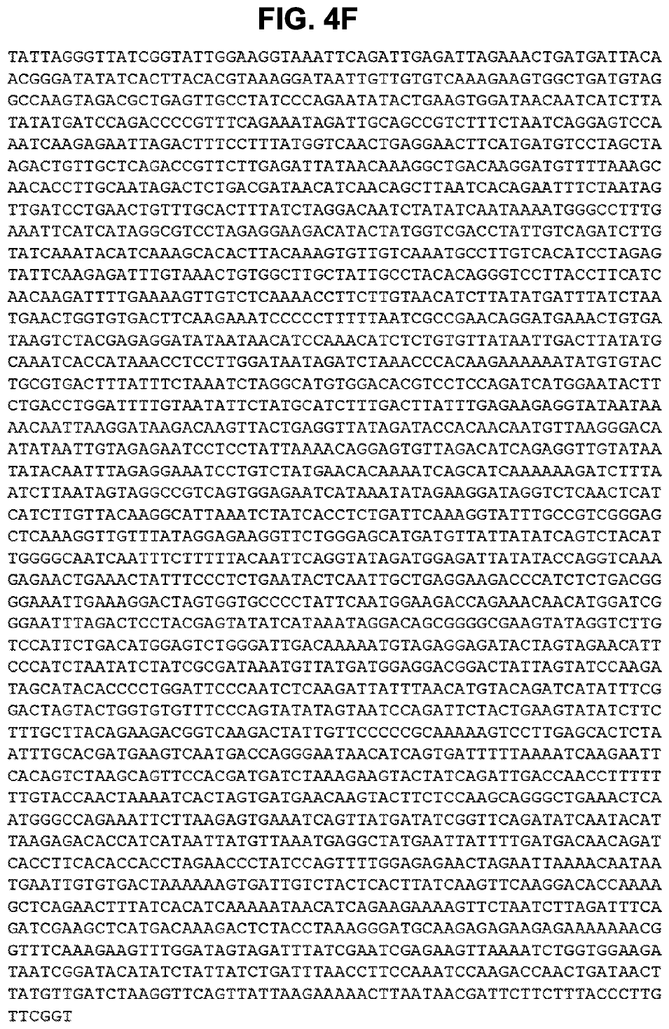

The disclosed NiV VRP include a recombinant Nipah henipavirus genome, wherein the recombinant Nipah henipavirus genome has a genetic inactivation, such as a deletion, in a nucleic acid sequence encoding the F protein (ΔF) such that functional mature F protein cannot be produced from the recombinant Nipah henipavirus genome. The Nipah henipavirus genome can be from a Bangladesh strain. The Nipah henipavirus genome can be from a Malaysian strain. An exemplary NiV Malaysia genome sequence, including the nucleic acid sequence encoding F protein, is provided in FIGS. 4A-4F (SEQ ID NO: 4).

The deletion in the F protein can be any deletion such that functional F protein is not produced. The F protein from NiV is initially synthesized as a single polypeptide precursor approximately 550 amino acids in length, designated F₀. In some embodiments, the genome of the VRP includes a deletion of the entire F protein. In one non-limiting example, the genome is from Nipah strain Malaysia, and includes a deletion of nucleotide 6366 to nucleotide 8707 (based on GENBANK® No. AF212302, as available on Dec. 2, 2021, incorporated herein by reference). In other embodiments, the genome of the VRP includes a deletion of at least about 1,000, at least about 1,250, at least about 1,500, at least about 1,750, at least about 2,000, or at least about 2,250 nucleotides of the nucleic acid sequence encoding the F protein. In other embodiments, the deletion is about 1,000 to about 2,300 nucleotides, about 1,250 to about 2,300 nucleotides, about 1,500 to about 2,300 nucleotides, about 1,750 to about 2,300 nucleotides, or about 2,000 to about 2,300 nucleotides of the nucleic acid sequence encoding the F protein. In further embodiments, the start codon is deleted. In other embodiments, the entire (2,342 nucleotides) sequence encoding the F protein is deleted.

F₀ includes an N-terminal signal peptide that directs localization to the endoplasmic reticulum, where the signal peptide is proteolytically cleaved. The remaining F₀ residues oligomerize to form a trimer and may be proteolytically processed by a cellular protease to generate two disulfide-linked fragments, F₁ and F₂. In some embodiments, the nucleic acid sequence encoding F₀ is deleted. In NiV F the cleavage site is located approximately between residues 109/110. The smaller of these fragments, F₂, originates from the N-terminal portion of the F₀ precursor (approximately residues 25-109). The larger of these fragments, F₁, includes the C-terminal portion of the F₀ precursor (approximately residues 110-550) including an extracellular/lumenal region (approximately residues 110-495), and a transmembrane and cytosolic regions (approximately residues 495-550). In other embodiments, the nucleic acid sequence encoding the C-terminal portion, or transmembrane and cytosolic regions, of F₁ is deleted. In another embodiment, the nucleic acid sequence encoding F₁ is deleted. The extracellular portion of the NiV F protein is the NiV Fectodomain, which includes the F₂ protein and the F₁ ectodomain. The fusion peptide is located at the N-terminal segment of the F₁ ectodomain, at approximately residues 110-122. In further embodiments the nucleic acid sequence encoding F₂ is deleted.

In other embodiments, the disclosed NiV VRP include a recombinant Nipah henipavirus genome, wherein the recombinant Nipah henipavirus genome has a genetic inactivation, such as a deletion, in a nucleic acid sequence encoding the G protein (ΔG) such that functional mature G protein cannot be produced from the recombinant Nipah henipavirus genome. The deletion in the G protein can be any deletion such that functional G protein is not produced. The NiV G gene is from nucleotides 8716 to nucleotide 11,261 in a wild-type NiV genome, with a coding sequence that is 1808 nucleotides in length (from nucleotide 8949 to 10757). In some embodiments, the coding sequence is deleted. In other embodiments, the start and/or termination sequence within this coding sequence are deleted. In other embodiments, the genome of the VRP includes a deletion of at least about 1,000, at least about 1,250, at least about 1,500, at least about 1,750, at least about 1800, at least about 2,000, or at least about 2,250 nucleotides of the nucleic acid sequence encoding the G protein. In other embodiments, the deletion is about 1,000 to about 2,300 nucleotides, about 1,250 to about 2,300 nucleotides, about 1,500 to about 2,300 nucleotides, about 1,750 to about 2,300 nucleotides, or about 2,000 to about 2,300 nucleotides of the nucleic acid sequence encoding the G protein. In other embodiments, the deletion is about 1,000 to about 1,808 nucleotides, about 1,250 to about 1,808 nucleotides, about 1,500 to about 1,808 nucleotides, or about 1,750 to about 1,808 nucleotides of the nucleic acid sequence encoding the G protein. The deletion can be at least 1,800 nucleotides of the nucleic acid sequence encoding the G protein. In further embodiments, the start codon is deleted. In other embodiments, the entire sequence encoding the G protein is deleted.

In some embodiments, the recombinant Nipah henipavirus genome has a genetic inactivation, such as a deletion, in a nucleic acid sequence encoding the G protein (ΔG) such that functional mature G protein cannot be produced from the recombinant Nipah henipavirus genome, and the recombinant Nipah henipavirus genome encodes a functional F protein.

In further embodiments, the disclosed NiV VRP include a recombinant Nipah henipavirus genome, wherein the recombinant Nipah henipavirus genome has a genetic inactivation, such as a deletion, in a nucleic acid sequence encoding the F protein (ΔF) such that functional mature F protein cannot be produced from the recombinant Nipah henipavirus genome and a genetic inactivation, such as a deletion, in a nucleic acid sequence encoding the G protein (ΔG) such that functional mature G protein cannot be produced from the recombinant Nipah henipavirus genome (ΔFΔG). Any of the deletions in the F and G proteins, as disclosed in the paragraphs above, can be utilized in this genome.

In some embodiments, the NiV VRP also includes a Nipah henipavirus envelope comprising F, G and M proteins of Nipah virus. In some embodiments, the Nipah henipavirus genome encodes the G and M proteins. Thus, in some embodiments, the Nipah henipavirus genome is from the Bangladesh strain, and the G and M proteins are also from the Bangladesh strain. In other embodiments, the Nipah henipavirus genome is from the Malaysian strain, and the G and M proteins are also from the Malaysian strain. In yet other embodiments, the G and/or M protein is from a HeV strain.

In other embodiments, the Nipah henipavirus genome is from the NiV Bangladesh strain, and the G protein is from a HeV strain. In other embodiments, the Nipah henipavirus genome is from the NiV Malaysian strain, and the G protein is from HeV. Thus, in some embodiments, the Nipah henipavirus genome encodes the G protein from a HeV strain. An exemplary G protein from an HeV strain is provided in GENBANK® Accession No. QDK64767.1, as available on Jan. 4, 2022, and an exemplary genome sequence for an HeV strain is provided in GENBANK® Accession No. MN062017, as available on Jan. 4, 2022, both incorporated by reference herein. An exemplary G protein sequence from HeV is also provided below:

(SEQ ID NO: 6)
MMADSKLVSLNNNLSGKIKDQGKVIKNYYGTMDIKKINDGLLDSK
ILGAFNTVIALLGSIIIIVMNIMIIQNYTRTTDNQALIKESLQSV
QQQIKALTDKIGTEIGPKVSLIDTSSTITIPANIGLLGSKISQST
SSINENVNDKCKFTLPPLKIHECNISCPNPLPFREYRPISQGVSD
LVGLPNQICLQKITSTILKPRLISYTLPINTREGVCITDPLLAVD
NGFFAYSHLEKIGSCTRGIAKQRIIGVGEVLDRGDKVPSMFMTNV
WTPPNPSTIHHCSSTYHEDFYYTLCAVSHVGDPILNSTSWTESLS
LIRLAVRPKSDSGDYNQKYIAITKVERGKYDKVMPYGPSGIKQGD
TLYFPAVGFLPRTEFQYNDSNCPIIHCKYSKAENCRLSMGVNSKS
HYILRSGLLKYNLSLGGDIILQFIEIADNRLTIGSPSKIYNSLGQ
PVFYQASYSWDTMIKLGDVDTVDPLRVQWRNNSVISRPGQSQCPR
FNVCPEVCWEGTYNDAFLIDRLNWVSAGVYLNSNQTAENPVFAVF
KDNEILYQVPLAEDDINAQKTITDCFLLENVIWCISLVEIYDTGD
SVIRPKLFAVKIPAQCSES

In some embodiments, the amino acid sequence of the G protein is at least about 95% identical to SEQ ID NO: 2, such as about 95%, 96%, 97%, 98% or 99% identical to SEQ ID NO: 2. In other embodiments, the amino acid sequence of the G protein includes the amino acid sequence of SEQ ID NO: 2 with at most 10 amino acid substitutions. In other embodiments, the amino acid sequence of the G protein includes the amino acid sequence of SEQ ID NO: 2 with at most 5 amino acid substitutions, such as 1, 2, 3, 4, or 5 conservative amino acid substitutions. In further embodiments, the amino acid sequence of the G protein includes, or consists of, SEQ ID NO: 2.

In other embodiments, the G protein is from another virus, such as a HeV. In some embodiments, the amino acid sequence of the G protein is at least about 95% identical to SEQ ID NO: 6, such as about 95%, 96%, 97%, 98% or 99% identical to SEQ ID NO: 6. In other embodiments, the amino acid sequence of the G protein includes the amino acid sequence of SEQ ID NO: 6 with at most 10 amino acid substitutions. In other embodiments, the amino acid sequence of the G protein includes the amino acid sequence of SEQ ID NO: 6 with at most 5 amino acid substitutions, such as 1, 2, 3, 4, or 5 conservative amino acid substitutions. In further embodiments, the amino acid sequence of the G protein includes, or consists of, SEQ ID NO: 6.

In more embodiments, the amino acid sequence of the M protein is at least about 95% identical to SEQ ID NO: 3, such as about 95%, 96%, 97%, 98% or 99% identical to SEQ ID NO: 3. In other embodiments, the amino acid sequence of the M protein includes the amino acid sequence of SEQ ID NO: 3 with at most 10 amino acid substitutions. In other embodiments, the amino acid sequence of the M protein includes the amino acid sequence of SEQ ID NO: 2 with at most 5 amino acid substitutions, such as 1, 2, 3, 4, or 5 conservative amino acid substitutions. In further embodiments, the amino acid sequence of the G protein includes, or consists of, SEQ ID NO: 2.

In more embodiments, the amino acid sequence of the M protein is at least about 95% identical to SEQ ID NO: 3, such as about 95%, 96%, 97%, 98% or 99% identical to SEQ ID NO: 3. In other embodiments, the amino acid sequence of the M protein includes the amino acid sequence of SEQ ID NO: 3 with at most 10 amino acid substitutions. In other embodiments, the amino acid sequence of the M protein includes the amino acid sequence of SEQ ID NO: 2 with at most 5 amino acid substitutions, such as 1, 2, 3, 4, or 5 conservative amino acid substitutions. In further embodiments, the amino acid sequence of the G protein includes, or consists of, SEQ ID NO: 2.

The NiV VRP includes the F protein, but the Nipah henipavirus genome does not encode functional F protein. Thus, for production of the NiV VRP, exogenous F protein is provided in trans, from the chromosome of a host cell or a vector included in a host cell. In some embodiments, the VRP includes a Malaysian Nipah henipavirus genome, and Malaysian F protein. In some embodiments, the VRP includes a Malaysian Nipah henipavirus genome, and Bangladesh F protein. In further embodiments, the VRP includes a Bangladesh Nipah henipavirus genome, and Bangladesh F protein. In further embodiments, the VRP includes a Bangladesh Nipah henipavirus genome, and Malaysian F protein.

In some embodiments, the amino acid sequence of the F protein is at least about 95% identical to SEQ ID NO: 1, such as about 95%, 96%, 97%, 98% or 99% identical to SEQ ID NO: 1. In other embodiments, the amino acid sequence of the F protein includes the amino acid sequence of SEQ ID NO: 1 with at most 10 amino acid substitutions. In other embodiments, the amino acid sequence of the F protein includes the amino acid sequence of SEQ ID NO: 1 with at most 5 amino acid substitutions, such as 1, 2, 3, 4, or 5 conservative amino acid substitutions. In further embodiments, the amino acid sequence of the F protein includes, or consists of, SEQ ID NO: 1.

In other embodiments NiV VRP includes the G protein, but the Nipah henipavirus genome does not encode functional G protein. Thus, for production of the NiV VRP, exogenous G protein is provided in trans, from the chromosome of a host cell or a vector included in a host cell. In some embodiments, the VRP includes a Malaysian Nipah henipavirus genome, and Malaysian G protein.

In some embodiments, the VRP includes a Malaysian Nipah henipavirus genome, and Bangladesh G protein. In further embodiments, the VRP includes a Bangladesh Nipah henipavirus genome, and Bangladesh G protein. In further embodiments, the VRP includes a Bangladesh Nipah henipavirus genome, and Malaysian F protein. In other embodiments, the VRP includes a Malaysian or Bangladesh Nipah henipavirus genome, and a HeV G protein.

In some embodiments, the amino acid sequence of the G protein is at least about 95% identical to SEQ ID NO: 2, such as about 95%, 96%, 97%, 98% or 99% identical to SEQ ID NO: 2. In other embodiments, the amino acid sequence of the G protein includes the amino acid sequence of SEQ ID NO: 2 with at most 10 amino acid substitutions. In other embodiments, the amino acid sequence of the G protein includes the amino acid sequence of SEQ ID NO: 2 with at most 5 amino acid substitutions, such as 1, 2, 3, 4, or 5 conservative amino acid substitutions. In further embodiments, the amino acid sequence of the G protein includes, or consists of, SEQ ID NO: 2.

In further embodiments NiV VRP includes the F and the G protein, but the Nipah henipavirus genome does not encode functional F or functional G protein. Thus, for production of the NiV VRP, exogenous F and G proteins are provided in trans, from the chromosome of a host cell or a vector included in a host cell. In some embodiments, the VRP includes a Malaysian Nipah henipavirus genome, and Malaysian F and G proteins. In some embodiments, the VRP includes a Malaysian Nipah henipavirus genome, and Bangladesh F and G proteins. In further embodiments, the VRP includes a Bangladesh Nipah henipavirus genome, and Bangladesh F and G proteins. In further embodiments, the VRP includes a Bangladesh Nipah henipavirus genome, and Malaysian F and G proteins.

In other embodiments, the VRP includes a Malaysian or Bangladesh Nipah henipavirus genome, a Malaysian or Bangladesh F protein, and a HeV G protein. In some non-limiting examples, the VRP can include: a) a Malaysian Nipah henipavirus genome, and Malaysian F protein, and a HeV G protein; b) a Malaysian Nipah henipavirus genome, and Bangladesh F protein, and a HeV G protein; c) a Bangladesh Nipah henipavirus genome,a Malaysian F protein, and a HeV G protein; or d) a Bangladesh Nipah henipavirus genome, a Bangladesh F protein, and a HeV G protein.

### Nucleic Acids, Host Cells, and Method for Producing VRP

Methods and composition are provided herein for producing a NiV VRP. The methods for producing NiV VRP include expressing NiV F protein from a host cell that is stably transfected with a nucleic acid molecule encoding the NiV F protein operably linked to a promoter; contacting the host cell with a disclosed NiV VRP, such that the cell in infected with the NiV genome that is includes a deletion of the F protein, and collecting NiV virus replicon particles produced by the host cell.

The compositions include a host cell stably expressing an NiV F protein, and vectors encoding the NiV F protein. In some embodiments, the amino acid sequence of the F protein is at least about 95% identical to SEQ ID NO: 1, such as about 95%, 96%, 97%, 98% or 99% identical to SEQ ID NO: 1. In other embodiments, the amino acid sequence of the F protein includes the amino acid sequence of SEQ ID NO: 1 with at most 10 amino acid substitutions. In other embodiments, the amino acid sequence of the F protein includes the amino acid sequence of SEQ ID NO: 1 with at most 5 amino acid substitutions, such as 1, 2, 3, 4, or 5 conservative amino acid substitutions. In further embodiments, the amino acid sequence of the F protein includes, or consists of, SEQ ID NO: 1.

In other embodiments, the compositions include a host cell stably expressing an NiV G protein, and vectors encoding the NiV G protein. In further embodiments, the amino acid sequence of the G protein is at least about 95% identical to SEQ ID NO: 2, such as about 95%, 96%, 97%, 98% or 99% identical to SEQ ID NO: 2. In other embodiments, the amino acid sequence of the G protein includes the amino acid sequence of SEQ ID NO: 2 with at most 10 amino acid substitutions. In other embodiments, the amino acid sequence of the G protein includes the amino acid sequence of SEQ ID NO: 2 with at most 5 amino acid substitutions, such as 1, 2, 3, 4, or 5 conservative amino acid substitutions. In further embodiments, the amino acid sequence of the G protein includes, or consists of, SEQ ID NO: 2. In other embodiments, the G protein is a HeV G protein.

In further embodiments compositions include a host cell stably expressing an NiV F protein and a NiV G protein, and vectors encoding both the NiV F protein and the NiV G protein.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

The nucleic acid molecule encoding the F protein can be codon-optimized for expression in human cells. An exemplary nucleic acid sequence comprises:

(SEQ ID NO: 5)
ATGGTGGTCATTCTGGACAAACGGTGTTACTGCAACCTGCTGATT
CTGATTCTGATGATTAGCGAGTGTAGTGTGGGCATTCTGCACTAC
GAGAAACTGTCCAAGATTGGACTGGTGAAAGGCGTCACTCGGAAG
TATAAAATCAAGTCTAACCCCCTGACCAAAGACATCGTGATTAAG
ATGATCCCTAACGTCAGTAATATGTCACAGTGCACAGGGTCCGTG
ATGGAGAACTACAAGACCAGACTGAATGGAATTCTGACACCCATC
AAAGGCGCCCTGGAAATCTATAAGAACAATACTCACGACCTGGTG
GGGGATGTCAGGCTGGCAGGAGTGATTATGGCAGGGGTCGCCATC
GGAATTGCCACTGCCGCTCAGATCACCGCTGGAGTGGCACTGTAC
GAGGCCATGAAGAACGCTGACAACATTAACAAGCTGAAGAGCAGC
ATCGAGTCTACCAATGAAGCTGTGGTCAAACTGCAGGAGACAGCA
GAAAAGACTGTGTACGTCCTGACAGCACTGCAGGACTATATCAAC
ACAAATCTGGTGCCTACTATCGATAAAATTAGTTGTAAGCAGACC
GAACTGTCACTGGACCTGGCTCTGAGCAAGTACCTGTCCGATCTG
CTGTTCGTGTTTGGCCCAAACCTGCAGGATCCCGTCTCTAATAGT
ATGACAATCCAGGCAATTAGCCAGGCCTTCGGCGGGAACTACGAG
ACCCTGCTGCGCACACTGGGCTATGCCACTGAGGACTTTGACGAT
CTGCTGGAATCAGATAGCATCACCGGGCAGATCATCTACGTGGAC
CTGTCTAGTTACTATATCATTGTGCGAGTCTACTTCCCAATTCTG
ACCGAGATCCAGCAGGCCTATATCCAGGAACTGCTGCCCGTGAGC
TTCAACAATGATAACTCTGAGTGGATCAGTATTGTGCCTAATTTT
ATTCTGGTCCGCAACACACTGATCAGCAATATCGAAATTGGCTTT
TGCCTGATTACTAAACGAAGCGTGATCTGTAATCAGGACTACGCC
ACCCCTATGACAAACAATATGCGGGAGTGCCTGACTGGAAGCACC
GAGAAGTGTCCCCGGGAGCTGGTGGTCTCAAGCCATGTGCCACGG
TTCGCCCTGAGCAACGGCGTGCTGTTTGCTAATTGCATCTCCGTC
ACCTGCCAGTGTCAGACCACAGGGCGCGCTATTTCCCAGTCTGGA
GAGCAGACACTGCTGATGATCGATAACACTACCTGTCCAACTGCA
GTGCTGGGCAATGTCATCATTAGCCTGGGCAAATACCTGGGGTCC
GTGAACTATAATTCTGAAGGAATCGCCATTGGCCCCCCTGTGTTC
ACCGACAAGGTCGATATTTCCTCTCAGATCAGTTCAATGAACCAG
TCACTGCAGCAGAGCAAAGACTACATCAAGGAGGCCCAGAGACTG
CTGGATACTGTGAATCCAAGCCTGATTTCCATGCTGTCTATGATC
ATTCTGTATGTGCTGAGTATCGCTTCACTGTGCATCGGGCTGATT
ACCTTCATCTCTTTTATCATTGTGGAGAAGAAAAGGAACACCTAC
AGTCGCCTGGAAGATCGGAGAGTGAGACCCACAAGCTCCGGCGAC
\CTGTACTATATTGGGACATGA

In some embodiments, the nucleic acid molecule encoding the F protein comprises, or consists of SEQ ID NO: 5.

Nucleic acids encoding an F protein can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Modifications can be made to a nucleic acid encoding the F protein without diminishing its biological activity. Some modifications can be made to facilitate the cloning or expression. Exemplary modifications include substituting termination codons, a methionine added at the amino terminus to provide an initiation, site, additional amino acids placed on either terminus to create conveniently located restriction sites, or additional amino acids (such as poly His) to aid in purification steps.

The nucleic acid molecule encoding the F protein can be operably linked to expression control sequences, such as a promoter. An expression control sequence operatively linked to a coding sequence is ligated such that expression of the coding sequence for the F protein is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Any promoter can be used that is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters include promoters isolated from mammalian genes, such as the immunoglobulin heavy chain, immunoglobulin light chain, T cell receptor, HLA-DQα and HLA-DQβ, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, neural cell adhesion molecule (NCAM), α1-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin.

The promoter can be either inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, α-2-macroglobulin, MHC class I gene h-2kb, HSP70, proliferin, tetracycline inducible, tumor necrosis factor, or thyroid stimulating hormone gene promoter. One example of an inducible promoter is the interferon inducible ISG54 promoter (see Bluyssen et al., Proc. Natl Acad. Sci. 92:5645-5649, 1995, herein incorporated by reference). In some embodiments, the promoter is a constitutive promoter that results in high levels of transcription upon introduction into a host cell in the absence of additional factors.

Optionally, transcription control sequences include one or more enhancer elements, which are binding recognition sites for one or more transcription factors that increase transcription above that observed for the minimal promoter alone, and also be operably linked to the polynucleotide encoding the promoter and/or the nucleic acid molecule encoding the F protein.

In some embodiments, the host cell is transformed with a vector encoding the F protein. Exemplary procedures sufficient to guide one of ordinary skill in the art through the production of a vector capable of expression in a host cell that includes a promoter, and/or a polynucleotide sequence encoding the F protein can be found, for example, in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Thus, the polynucleotides encoding the NiV F protein can include a recombinant DNA encoding an F protein which is incorporated into a vector (such as an expression vector) into an autonomously replicating plasmid or virus or any vector that incorporates into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA. The nucleic acid sequence including the expression control sequences operably linked to the nucleic acid molecule encoding the F protein can be integrated into the chromosome of a host cell.

DNA sequences encoding the NiV F protein can be expressed in vitro by DNA transfer into a suitable host cell. The cell may be eukaryotic, such as a mammalian host cell, for example a human host cell. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells. In one embodiment, the host cell is a Vero cell, a CHO cell, a NCI-H358 cell, or a HEK-293 T cell. In more embodiments, the cells are modified to express Ephrin B 2.

Transformation of a host cell with recombinant DNA can be carried out by conventional techniques. When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

A nucleic acid molecule encoding the F protein can be included in a viral vector, for example, for expression in a host cell. Viral vectors include polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739).

### Immunogenic Compositions

NiV VRP or immunogenic compositions comprising these NiV VRP, can be administered to a subject by any of the routes normally used for introducing virus or virus particles into a subject. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Immunogenic compositions are administered in any suitable manner, such as with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. Actual methods for preparing administrable compositions are described in more detail in such publications as Remingtons Pharmaceutical Sciences, 19^{th} Ed., Mack Publishing Company, Easton, Pennsylvania, 1995.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The NiV VRP and immunogenic compositions including these VRP can be administered alone or in combination with other therapeutic agents to enhance antigenicity. The NiV VRP and immunogenic compositions including these VRP can be formulated without an adjuvant. The NiV VRP and immunogenic compositions including these VRP can be administered in the absence of an adjuvant.

In some embodiments, the disclosed VRP can be administered with an adjuvant, such as Freund incomplete adjuvant or Freund's complete adjuvant. These adjuvants can be included in the immunogenic compositions.

Adjuvants, such as aluminum hydroxide (ALHYDROGEL®, available from Brenntag Biosector, Copenhagen, Denmark and AMPHOGEL®, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), TLR agonists (such as TLR-9 agonists), among many other suitable adjuvants well known in the art, can be included in the compositions. Suitable adjuvants are, for example, toll-like receptor agonists, alum, AlPO₄, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances, the adjuvant formulation is a mineral salt, such as a calcium or aluminum (alum) salt, for example calcium phosphate, aluminum phosphate or aluminum hydroxide. In some embodiments, the adjuvant includes an oil and water emulsion, e.g., an oil-in-water emulsion (such as MF59 (Novartis) or AS03 (GlaxoSmithKline). One example of an oil-in-water emulsion comprises a metabolisable oil, such as squalene, a tocol such as a tocopherol, e.g., alpha-tocopherol, and a surfactant, such as sorbitan trioleate (Span 85) or polyoxyethylene sorbitan monooleate (Tween 80), in an aqueous carrier.

Optionally, one or more cytokines, such as IL-2, IL-6, IL-12, RANTES, GM-CSF, TNF-α, or IFN-γ, one or more growth factors, such as GM-CSF or G-CSF; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). An adjuvant can be administered systemically (or locally) to the host, either in the same compositions, simultaneously in a different composition, or sequentially.

In some embodiment a disclosed composition can be combined with other pharmaceutical products (e.g., vaccines) which induce protective responses to other agents. For example, a disclosed NiV VRP can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age). As such, a disclosed NiV VRP described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and/or rotavirus.

In some embodiments, the composition can be provided as a sterile composition. Typically, the amount of VRP in each dose of the immunogenic composition is selected as an amount which induces an immune response without significant, adverse side effects. In some embodiments, the composition can be provided in unit dosage form for use to induce an immune response in a subject, for example, to inhibit NiV infection in the subject. A unit dosage form contains a suitable single preselected dosage for administration to a subject, or suitable marked or measured multiples of two or more preselected unit dosages, and/or a metering mechanism for administering the unit dose or multiples thereof.

### Methods of Inducing an Immune Response

The immunogenic composition including the NiV VRP can be administered to a subject to induce an immune response to NiV in the subject. The disclosed VRP and immunogenic compositions can be used for example, to prevent or inhibit NiV infection. The immune response can be a protective immune response, for example a response that inhibits subsequent infection with NiV. Elicitation of the immune response can also be used to treat or inhibit NiV infection and illnesses associated therewith. The disclosed methods can reduce a symptom of a NiV infection and/or reduce viral titer.

A subject can be selected for treatment that has, or is at risk for developing NiV infection, for example because of exposure or the possibility of exposure to NiV. Following administration of a disclosed immunogen, the subject can be monitored for the NiV infection or symptoms associated therewith, or both.

In other embodiments, the immunogenic composition including the NiV VRP can be administered to a subject to induce an immune response to HeV in the subject, such as when the VRP includes the HeV G protein. The disclosed VRP and immunogenic compositions can be used for example, to prevent or inhibit a HeV infection. The immune response can be a protective immune response, for example a response that inhibits subsequent infection with HeV. Elicitation of the immune response can also be used to treat or inhibit the HeV infection and illnesses associated therewith. The disclosed methods can reduce a symptom of a HeV infection and/or reduce viral titer.

A subject can be selected for treatment that has, or is at risk for developing a HeV infection, for example because of exposure or the possibility of exposure to HeV. Following administration of a disclosed immunogen, the subject can be monitored for the HeV infection or symptoms associated therewith, or both.

Typical subjects intended for treatment with the therapeutics and methods of the present disclosure include humans and domestic animals such as pigs. In a particular example, the subject is a human. In other examples, the subject is a veterinary subject, such as a pig or hamster. In several embodiments, the subject is a human subject that is seronegative for NiV specific antibodies. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize NiV infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogenic composition can be for prophylactic or therapeutic purpose. When provided prophylactically, the immunogenic composition can be provided in advance of any symptom, for example in advance of infection. The prophylactic administration serves to prevent or ameliorate any subsequent infection. In some embodiments, the methods can involve selecting a subject at risk for contracting NiV infection, and administering a therapeutically effective amount of a disclosed immunogenic composition to the subject. The immunogenic composition can be provided prior to the anticipated exposure to NiV so as to attenuate the anticipated severity, duration or extent of an infection and/or associated disease symptoms, after exposure or suspected exposure to the virus, or after the actual initiation of an infection. When provided therapeutically, the disclosed immunogenic composition are provided at or after the onset of a symptom of NiV infection, or after diagnosis of NiV infection. Treatment of NiV by inhibiting NiV replication or infection can include delaying and/or reducing signs or symptoms of NiV infection in a subject. In some examples, treatment using the methods disclosed herein prolongs the time of survival of the subject.

In other embodiments, the methods can involve selecting a subject at risk for contracting HeV infection, and administering a therapeutically effective amount of a disclosed immunogenic composition to the subject, wherein the VRP includes the HeV G protein. The immunogenic composition can be provided prior to the anticipated exposure to HeV so as to attenuate the anticipated severity, duration or extent of an infection and/or associated disease symptoms, after exposure or suspected exposure to the virus, or after the actual initiation of an infection. When provided therapeutically, the disclosed immunogenic composition are provided at or after the onset of a symptom of the HeV infection, or after diagnosis of a HeV infection. Treatment of HeV by inhibiting HeV replication or infection can include delaying and/or reducing signs or symptoms of a HeV infection in a subject. In some examples, treatment using the methods disclosed herein prolongs the time of survival of the subject.

In some embodiments, administration of a disclosed immunogenic composition to a subject can elicit the production of an immune response that is protective against serious lower respiratory tract disease, such as pneumonia and bronchiolitis, or croup, when the subject is subsequently infected or re-infected with a wild-type NiV. While the naturally circulating virus may still be capable of causing infection, particularly in the upper respiratory tract, there can be a reduced possibility of rhinitis as a result of the vaccination and a possible boosting of resistance by subsequent infection by wild-type virus. Following administration of a disclosed immunogenic composition, there are detectable levels of host engendered serum and secretory antibodies which are capable of neutralizing homologous (of the same subgroup) wild-type virus in vitro and in vivo. In many instances the host antibodies will also neutralize wild-type virus of a different, non-vaccine subgroup.

The immunogenic compositions are provided to a subject in an amount effective to induce or enhance an immune response in the subject, preferably a human. The actual dosage will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to NiV F protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

In some embodiments of the methods, the immunogenic composition is administered in a single dose. In another embodiment, the immunogenic composition is administered in multiple doses, such as two, three or four doses. When administered in multiple doses, the time period between doses can vary. In some cases, the time period is days, weeks or months. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., inhibition of NiV infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the VRP is administered at a dose of about 10² to about 10⁶ TCID₅₀. In some examples, the VRP is administered at a dose of about 10³ to about 10⁵ TCID₅₀. In particular examples, the VRP is administered at a dose of about 10², about 10³, about 10⁴, about 10⁵ or about 10⁶ TCID₅₀. An optimal amount for a particular composition can be ascertained by standard studies involving observation of antibody titers and other responses in subjects. It is understood that a therapeutically effective amount of a disclosed immunogenic composition, can include an amount that is ineffective at eliciting an immune response by administration of a single dose, but that is effective upon administration of multiple dosages, for example in a prime-boost administration protocol. In other embodiments, a therapeutically effective amount is the amount sufficient to induce an immune response as a single dose.

Upon administration of a disclosed immunogenic composition, the immune system of the subject typically responds to the immunogenic composition by producing antibodies specific for the virus. Such a response signifies that an immunologically effective dose was delivered to the subject.

For each particular subject, specific dosage regimens can be evaluated and adjusted over time according to the individual need and professional judgment of the person administering or supervising the administration of the immunogenic composition. The dosage and number of doses will depend on the setting, for example, in an adult or anyone primed by prior NiV infection or immunization, a single dose may be a sufficient booster. In naïve subjects, in some examples, at least two doses would be given, for example, at least three doses. In some embodiments, an annual boost is given, for example, along with an annual influenza vaccination.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, a NiV F protein, a NiV G protein, a HeV G protein, and/or a NiV M protein.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, hamster, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Administration of an immunogenic composition that elicits an immune response to reduce or prevent an infection, can, but does not necessarily completely, eliminate such an infection, so long as the infection is measurably diminished. In some embodiments, administration of an effective amount of the immunogenic composition can decrease the NiV infection, for example, as measured by infection of cells, or by number or percentage of subjects infected by NiV by a desired amount, for example by at least 10%, at least 20%, at least 50%, at least 60%, at least 70%, at least 80%, at least 90%, at least 95%, at least 98%, or even at least 100% of detectable NiV infection, as compared to a suitable control. In other embodiments, administration of an effective amount of the immunogenic composition can decrease the symptoms of the NiV infection, for example by at least 10%, at least 20%, at least 50%, at least 60%, at least 70%, at least 80%, at least 90%, at least 95%, at least 98%, or even at least 100%, as compared to a suitable control. The control can be the amount in an untreated subject, or the amount in the same subject prior to treatment.

In more embodiments, administration of an effective amount of the immunogenic composition can decrease the HeV infection, for example, as measured by infection of cells, or by number or percentage of subjects infected by HeV by a desired amount, for example by at least 10%, at least 20%, at least 50%, at least 60%, at least 70%, at least 80%, at least 90%, at least 95%, at least 98%, or even at least 100% of detectable HeV infection, as compared to a suitable control. In other embodiments, administration of an effective amount of the immunogenic composition can decrease the symptoms of the HeV infection, for example by at least 10%, at least 20%, at least 50%, at least 60%, at least 70%, at least 80%, at least 90%, at least 95%, at least 98%, or even at least 100%, as compared to a suitable control. The control can be the amount in an untreated subject, or the amount in the same subject prior to treatment.

In further embodiments, administration of an effective amount of the agent can decrease a NiV infection (for example, as measured by infection of cells, or by number or percentage of subjects infected by NiV by a desired amount, for example by at least 10%, at least 20%, at least 50%, at least 60%, at least 70%, at least 80%, at least 90%, at least 95%, at least 98%, or even at least 100% (elimination or prevention) of detectable NiV infection, as compared to a suitable control. In other embodiments, administration of an effective amount of the agent can decrease symptoms of subjects infected by NiV by a desired amount, for example by at least 10%, at least 20%, at least 50%, at least 60%, at least 70%, at least 80%, at least 90%, at least 95%, at least 98%, or even at least 100% of detectable NiV infection, as compared to a suitable control. The control can be a similar value in untreated subjects. Thus, the administration of the effective amount can reduce a NiV infection in a population.

In other embodiments, administration of an effective amount of the agent can decrease a HeV infection (for example, as measured by infection of cells, or by number or percentage of subjects infected by HeV by a desired amount, for example by at least 10%, at least 20%, at least 50%, at least 60%, at least 70%, at least 80%, at least 90%, at least 95%, at least 98%, or even at least 100% (elimination or prevention) of detectable HeV infection, as compared to a suitable control. In other embodiments, administration of an effective amount of the agent can decrease symptoms of subjects infected by HeV by a desired amount, for example by at least 10%, at least 20%, at least 50%, at least 60%, at least 70%, at least 80%, at least 90%, at least 95%, at least 98%, or even at least 100% of detectable HeV infection, as compared to a suitable control. The control can be a similar value in untreated subjects. Thus, the administration of the effective amount can reduce a HeV infection in a population.

In some embodiments, administration of a therapeutically effective amount of one or more of the disclosed immunogenic composition(s) to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays. In some embodiments, the serum neutralization activity can be assayed using a panel of NiV pseudoviruses. Desirable outcomes also include the development of a T cell response, such as induction of an antigen-specific CD4 and/or CD8+ T cell response. Clinical benefit can be measured as a reduction in the titer of virus or infectious particles in blood or in a tissue biopsy, or a limitation in the progression of the disease.

In some embodiments, the subject is further administered a second anti-viral agent. Exemplary anti-viral agents include, but are not limited to, ribavirin, an IgG, or a monoclonal antibody. Additional agents include, but are not limited to, Remdesivir and the monoclonal antibody m102.4.

### EXAMPLES

The following examples are provided to illustrate certain particular features and/or embodiments. These examples should not be construed to limit the disclosure to the particular features or embodiments described.

### Example 1

### Generation of a Cell Line Constituently Expressing NiV Fusion Protein (F)

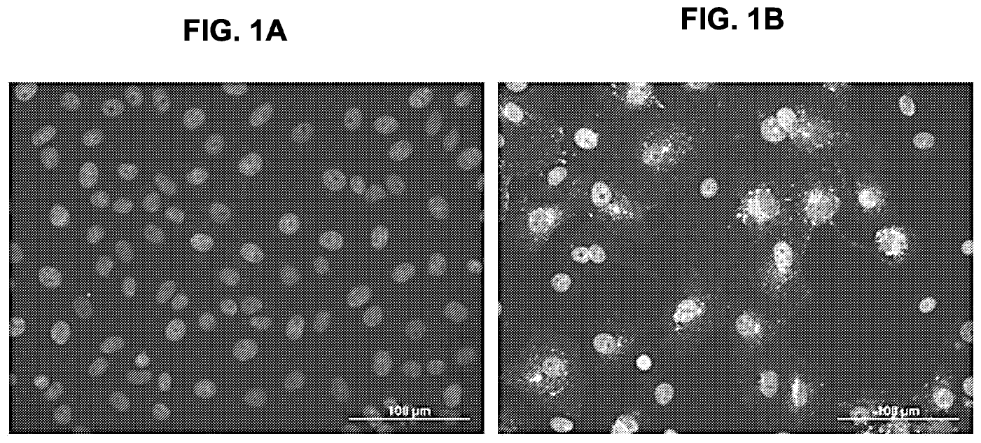

The coding sequence (CDS) for a codon optimized form of the fusion protein (Fco) from Nipah strain Malaysia (Genbank AF212302) was inserted into the genome of Vero cells using a commercial kit (Invitrogen FlpIn). This kit uses specific plasmids in a two-step process to stably introduce foreign genes into cells. Stable expression of Fco was confirmed using specific anti-NiV antibodies (see FIGS. 1A and 1B). These cells were termed Vero-NiVFco cells.

### Example 2

### Rescue of a Recombinant NiV-Malaysia Virus Lacking the Fusion Protein (F) Gene

Using the existing reverse genetics system for NiV-M, entire F gene (gene start—CDS—gene end) was excised from the genomic rescue plasmid using excision PCR methods. The sequence of the modified plasmid was assessed by restriction digest and NGS methods, confirmed the complete removal of the F gene with no additional nucleotide changes to the NiV genome.

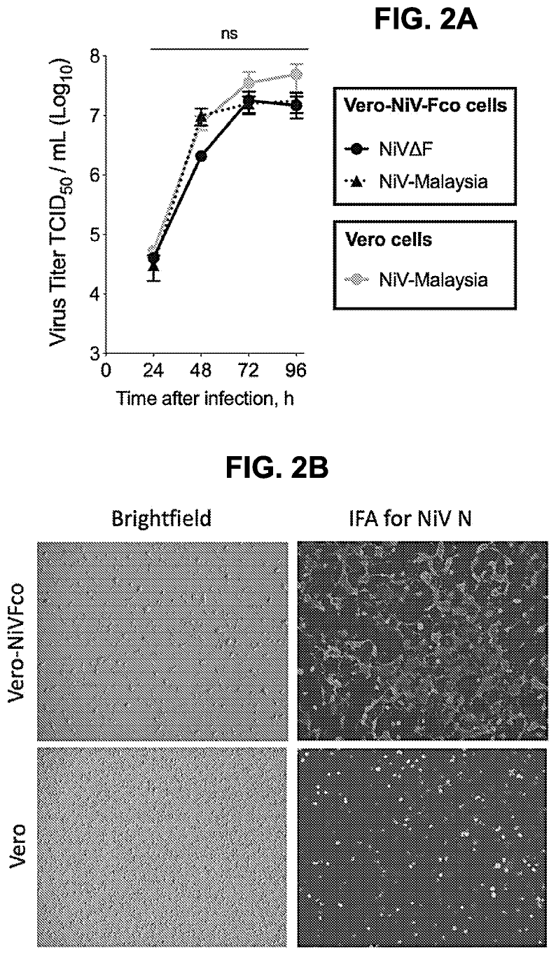

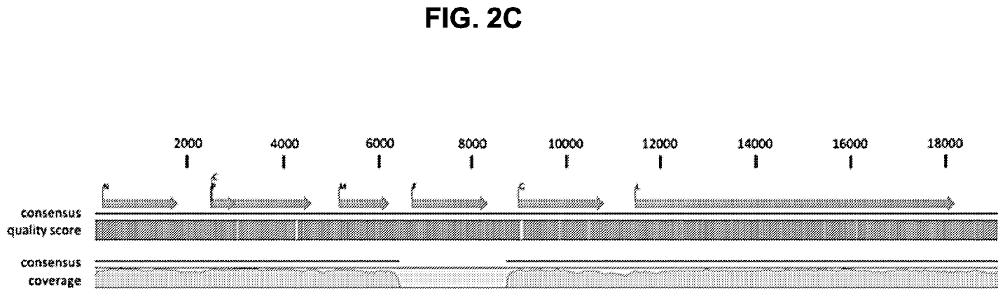

To rescue the NiVΔF, BSR-T7/5 cells were first transfected with the modified genomic plasmid alongside helper expression plasmids for NiV N-, L-, P, and F-proteins. Two days post transfection (dpt), the transfected cells were overlaid with Vero-NiVFco cells and monitored daily for cytopathic effect (CPE). At 7 dpt (5 days post overlay) clear signs of CPE consistent with NiV infection (i.e. syncytia formation) was observed. Cell culture supernatant was removed, clarified by low-speed centrifugation and used to infect both Vero-NiVFco and Vero cells. At 2 days post infection (dpi) widespread CPE was observed in the Vero-NiVFco cells, whereas the Vero monolayer remained largely intact with only those individual cells infected with NiVΔF showing signs of virus infection (FIG. 2A). Immunofluorescent staining with anti-NiV antibodies confirmed that the CPE was due to NiVΔF propagation in the cell monolayer (FIG. 2B). NGS analysis of the NiVΔF VRP stock confirmed the absence of the F gene in the NiVΔF genome (FIG. 2C).

### Example 3

### Efficacy of NiVΔF Vaccination in the Syrian Hamster Model of NiV-M Disease

Hamsters (groups of 8 or 10, split equally between males and females) were vaccinated intranasally (IN) with 10⁶ TCID₅₀ of NiVΔF, with total vaccination period prior to challenge being either 28, 14, 7, 3, or 1 days. One further group was double vaccinated IN with 10⁶ TCID₅₀ of NiVΔF both 3 and 1 day prior to challenge. Hamsters were then challenged with wildtype NiV-M either IN (10⁶ TCID₅₀) or intraperitoneally (IP, 10⁴ TCID₅₀). All hamsters were 7-8 weeks old at time of challenge. Throughout the vaccination period, no weight loss, fever, of clinical signs consistent with NiV infection were observed.

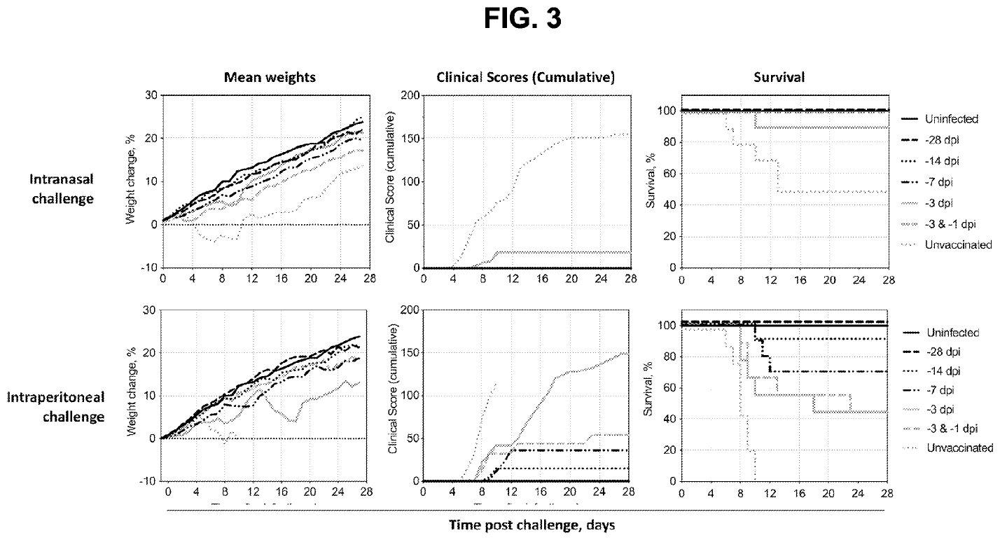

In the IN challenged groups, hamsters vaccinated once at 28, 14, 7, or 3 days prior to challenge, or twice at 3 and 1 days prior to challenge, demonstrated no weight loss, fever, or clinical signs (FIG. 3). All animals in these groups survived until end of study (28 days post challenge). In the −3, and −1 groups, clinical signs and death was observed in 1 and 5 animals, respectively. In unvaccinated animals, clinical signs were observed in 100% of animals, with 50% succumbing to infection. In the IP challenged groups, hamsters vaccinated 28 days prior to challenge again demonstrated no weight loss, fever, or clinical signs, with 100% of the animals surviving until end of study. In the animals vaccinated 14, 7, and 3 & 1, days before challenge, 1, 3, and 5 animals, respectively, demonstrated clinical signs and succumbed to infection. All remaining animals displayed no signs of infection and survived until end of study. In the animals vaccinated 3 days prior to challenge, 6 displayed signs of infection and 5 succumbed to infection. In unvaccinated animals, 100% succumbed to infection.

### Example 4

### Safety of NiVΔF Vaccination in in Three Highly Susceptible Small Animal Model of NiV Disease

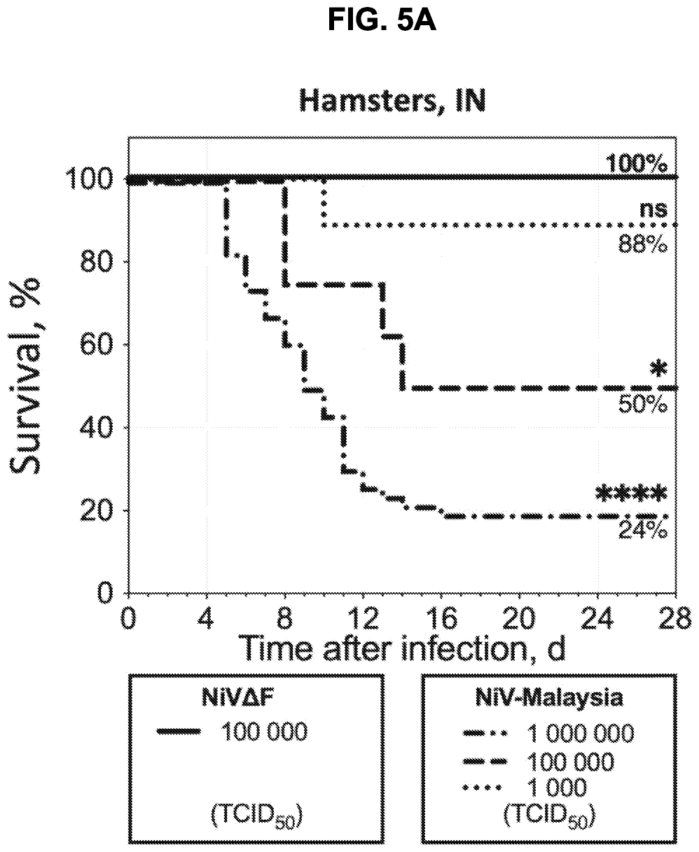

Using the Syrian hamster disease model, animals (n=20, 5-7 weeks old) intranasally (IN) inoculated with 10⁶ TCID₅₀ NiVΔF closely matched all clinical parameters of mock vaccinated hamsters: no significant changes in weights or body temperature; no clinical signs apparent; and all survived until completion of the 28-day study period. Conversely, IN inoculation with 10⁶ TCID₅₀ wildtype NiV (n=46, 5-7 weeks old, historical in-house data) resulted in 76% mortality [p=<0.0001] with all animals exhibiting clinical signs at some point during the study (FIG. 5A). Infection also was compared to wildtype NiV in the highly sensitive suckling mouse model. Groups of 9-14 animals (2-3 day old) were inoculated intracerebrally (IC) and monitored daily for lethality and clinical signs. A 10⁵ TCID₅₀ IC inoculation with NiVΔF resulted in 100% survival with no clinical signs (n=12). All mice inoculated with 10⁶ to 10² TCID₅₀ of wildtype NiV succumbed by 4 dpi [p=<0.0001], with reduced mortality (66% ) [p=0.0008] seen at 10¹ TCID₅₀ (FIG. 5B). Finally, a high safety profile of NiVΔF was demonstrated in IFNAR^{-/-} mice, with groups of 8 mice inoculated IP with NiVΔF (10⁶ TCID₅₀) or NiV (10⁶ or 10⁴ TCID₅₀). At a dose of 10⁴ TCID₅₀, 7 of 8 mice (88%) had clinical signs and 4 of 8 (50%) succumbed to disease. At the higher dose of 10⁶ TCID₅₀, 8 of 8 mice (100%) had clinical signs and 63% succumbed to disease. In contrast, mice inoculated with NiVΔF at the higher dose of 10⁶ TCID₅₀ all survived and had no apparent clinical signs throughout the 28 day study period (FIG. 5C).

### Example 5

### NiVΔF Demonstrates a Non-Spreading Phenotype in in the Highly Susceptible Syrian Hamsters Small Animal Model of NiV Disease

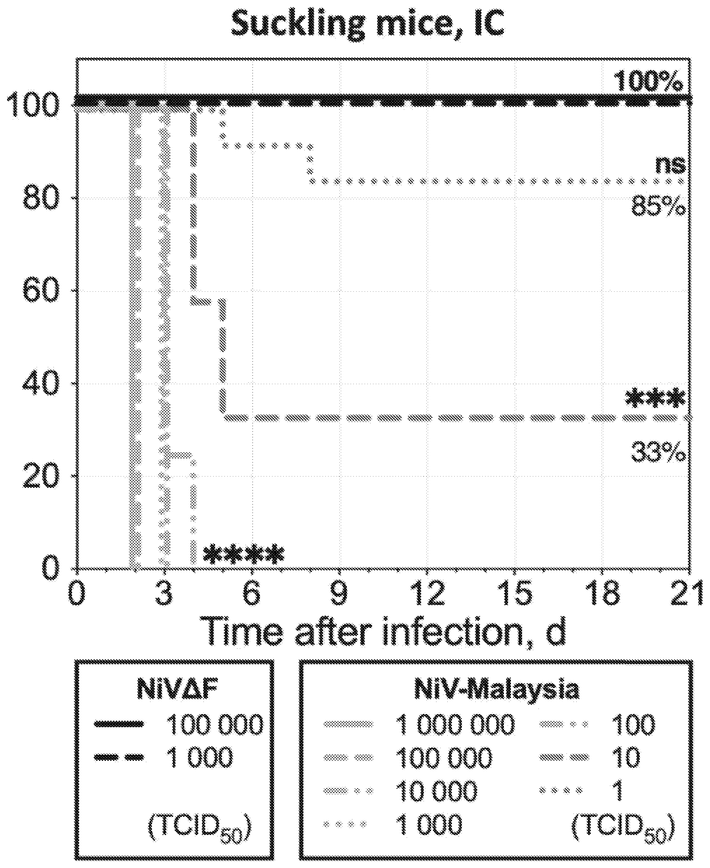

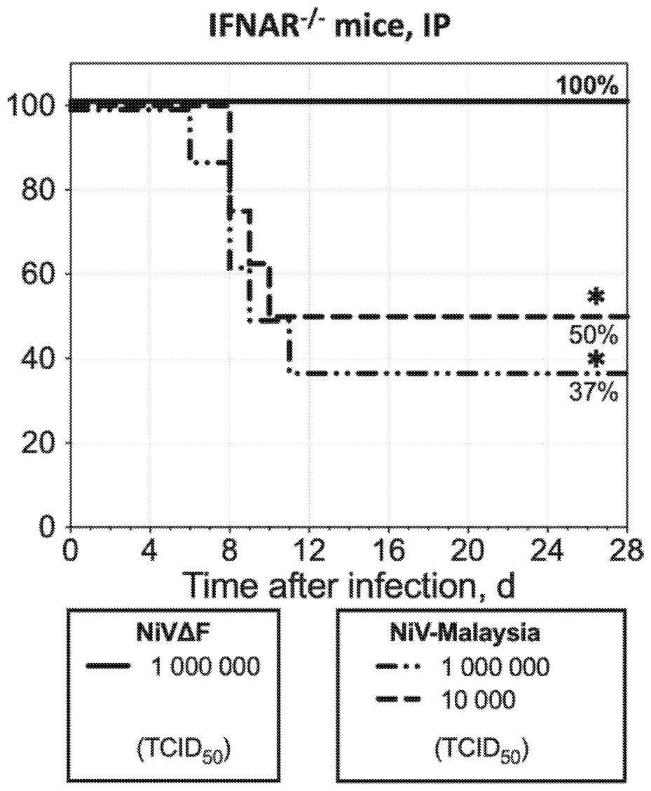

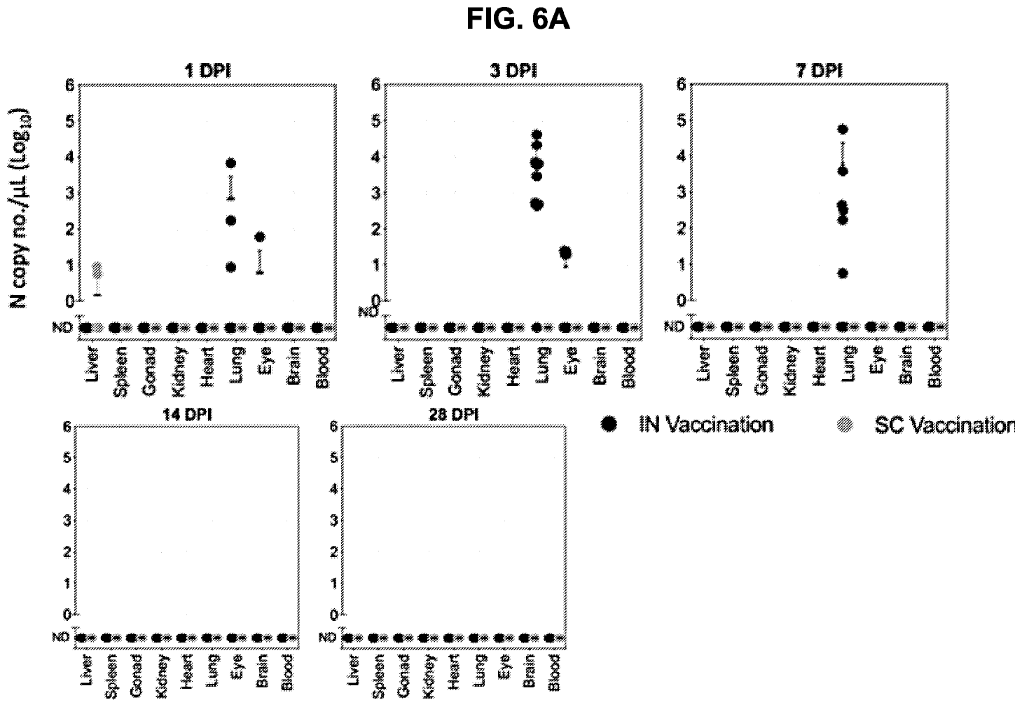

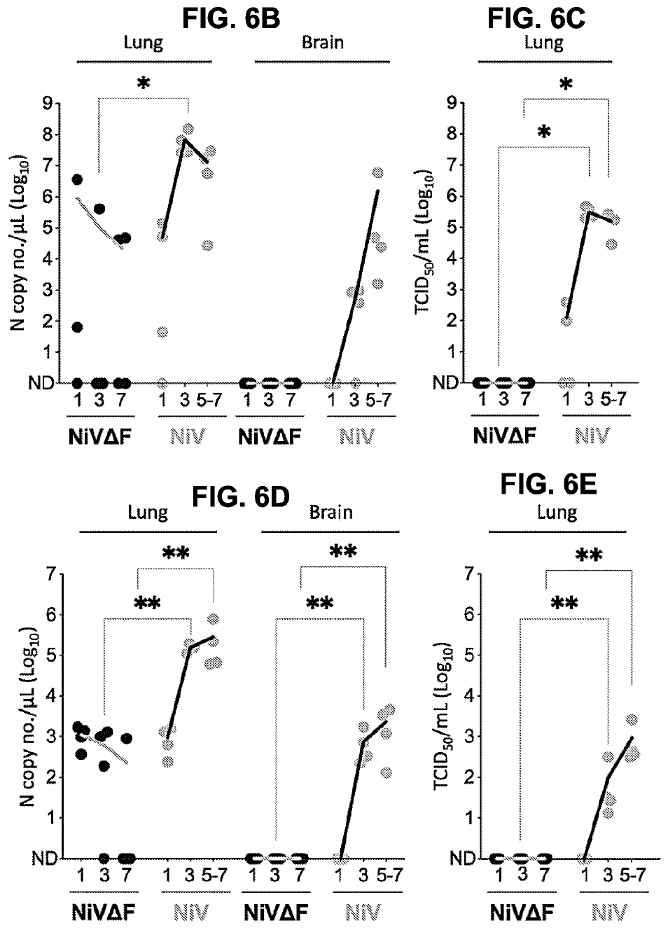

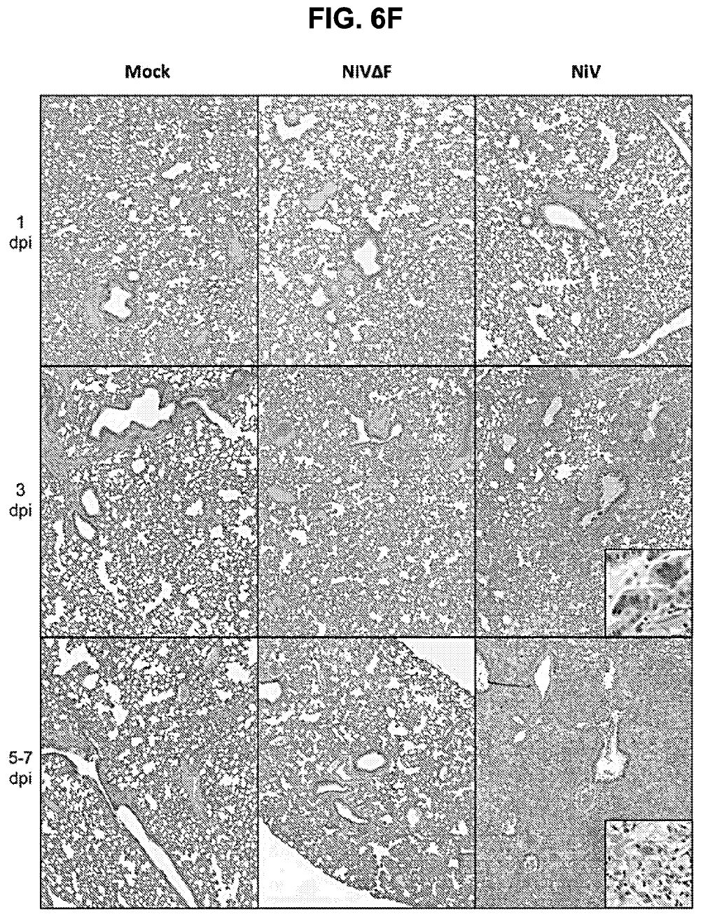

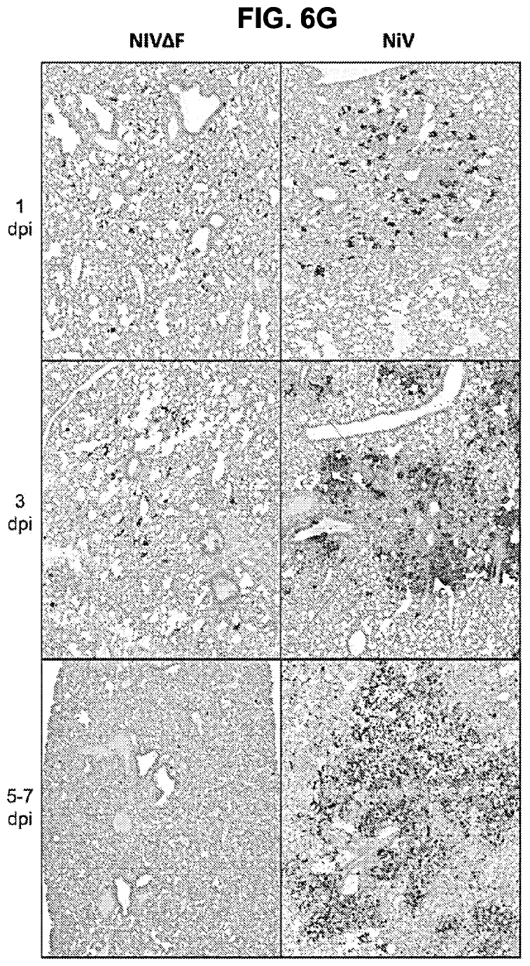

Two experiments were performed to confirm the non-spreading non-infectious nature of NiVΔF in vivo. First, groups of hamsters were vaccinated either IN or subcutaneously (SC) with 10⁶ TCID₅₀ of NiVΔF and serially euthanized at 1, 3, 7, 14, or 28 days post-inoculation (dpi; n=10 per route each timepoint). Independent of route, NiVΔF delivery resulted in limited and transient tissue dissemination (FIG. 6A). In IN exposed animals, NiVΔF RNA was detected by RT-qPCR in the lungs up to one week post-inoculation. Low levels (<100 genome copies) of NiVΔF were detected in the eyes of a minority of animals at 1 and 3 dpi. In SC challenged animals, aside from low levels (<10 genome copies) in 2 animals at 1 dpi in the liver, no NiVΔF was detected.

Next, hamsters were IN-inoculated with NiVΔF, wildtype NiV or DMEM (negative control) and groups (n=4 each) were serially euthanized at 1, 3, and 7 dpi (or earlier if animals met euthanasia criteria due to NiV disease). Tissues were collected for vRNA, virus isolation, and histopathological evaluation; and swab specimens (oral, rectal) were collected to detect mucosal shedding as characterized in the model. NiVΔF vRNA was detected in lung but not brain, and was not isolated from either tissue, whereas NiV was detected in both tissues and isolated from lung (FIG. 6B, 6C). Viral RNA in oropharyngeal samples taken from NiVΔF-inoculated hamsters at 4 and 7 dpi were significantly [p=0.029] lower compared to vRNA in NIV inoculated hamsters, and, as expected, NiVΔF could not be isolated. In rectal swabs, no NiVΔF RNA was detected post vaccination compared [p=0.029] to rising levels detected at 3 and 7 dpi in NiV infected hamsters (FIG. 6D, 6E).

Histopathological examination of tissues collected at 1, 3, and 7 dpi indicated the absence of pathology in all NiVΔF tissues examined (liver, spleen, gonad, kidney, heart, lung, eye, and brain) at all timepoints. In contrast, NiV infection resulted in inflammation and progressive bronchointerstitial pneumonia with syncytial cells characteristic of paramyxoviral infection by 7 dpi in all except 1 animal (FIG. 2F). This one animal did not develop pneumonia but had a focus of neuronal degeneration and meningeal inflammation in the olfactory bulb of the brain. In situ hybridization was used to examine vRNA over time in lung and select other tissues. NiVΔF RNA was detected in lung at 1 dpi and decreased by 7 dpi. In contrast, NiV RNA was detected at relatively higher levels at 1 dpi, and in situ hybridization (ISH) demonstrated increased staining in the pneumonic lungs at later timepoints (FIG. 2G). One 7 dpi NiV had focal ISH staining in extrapulmonary tissues without overt pathologic changes (brain, liver, spleen, kidney, adrenal gland, renal lymph node), and the 7 dpi animal with focal brain pathology had extensive viral RNA in the rostral brain tissue.

### Example 6

### Parenteral or Mucosal Delivery of NiVΔF Elicits a Robust Non-Neutralizing Antibody Response

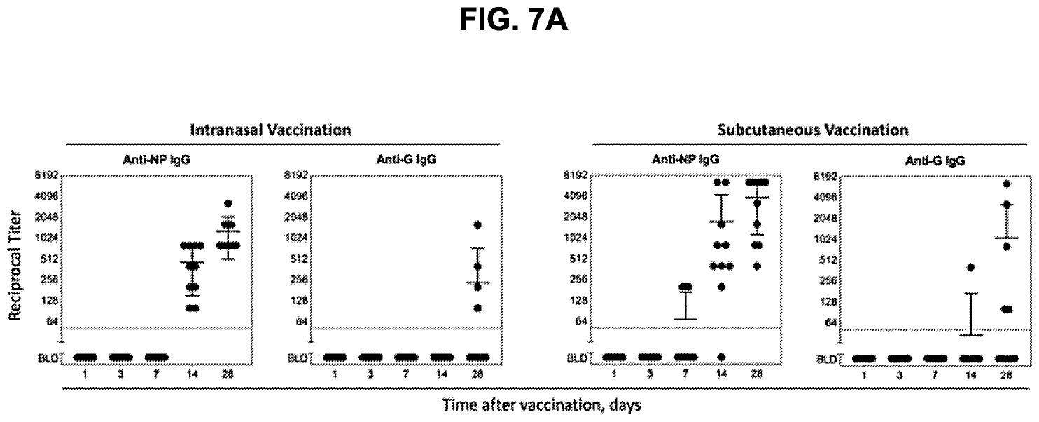

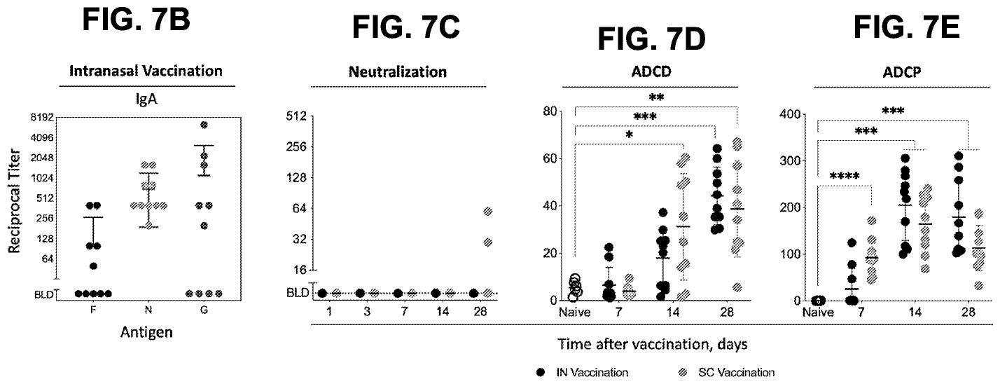

To characterize NiVΔF immunogenicity following peripheral and mucosal delivery, groups of hamsters were vaccinated either IN or SC with 10⁶ TCID₅₀ and serially euthanized at 1, 3, 7, 14, or 28 days post-inoculation (dpi; n=10 per route each timepoint). Antibody responses to NiVΔF were noted beginning at 7 dpi (SC-inoculated) and 14 dpi (IN-inoculated). A minority of animals vaccinated SC developed an anti-N IgG response at 7 dpv, and all but 1 animal had an anti-N IgG response 14 dpv with both vaccination routes (FIG. 7A). The anti-G IgG response was less pronounced with only 40-50% of animals developing a detectable response by 28 dpv. In all cases, the anti-N response was stronger than the anti-G response. The serum IgA response was examined at 28 dpv in IN vaccinated animals. As with IgG, all animals (n=10) demonstrated an anti-N IgA response, but 6 of 10 and 5 of 10 also had detectable anti-G and anti-F IgA, respectively (FIG. 7B). Interestingly, both vaccination routes resulted in almost a complete lack of antibody neutralizing activity against NiV-Malaysia and NiV-Bangladesh strains, with only 2 animals (both SC vaccinated) developing low-level neutralizing antibody response (FIG. 7C).

To investigate NiVΔF elicited antibody function in the absence of neutralization, Fc-effector function assays were performed. In an antibody-dependent complement deposition

(ADCD) assay examining immunoglobulins targeting NiV nucleoprotein (N), an increased response correlated to the length of vaccination period, with significant levels of complement deposition seen 28 days post vaccination in both IN [p=0.0003] and SC [p=0.0018] routes over mock vaccinated animals FIG. 7D). Phagocytic activity of antibodies was detected at 7, 14, and 28 days post vaccination with NiVΔF (FIG. 7E). These data demonstrate that after both IN and SC vaccination with NiVΔF, a broad adaptive immune response develops, with multiple immunoglobulin classes targeting N, F, and G detected. Although these antibodies appear non-neutralizing, Fc-effector functions such as complement deposition were associated with their presence.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A Nipah henipavirus virus replicon particle comprising:
a recombinant Nipah henipavirus genome, wherein the recombinant Nipah henipavirus genome comprises a deletion in a nucleic acid sequence encoding the F protein such that functional mature F protein cannot be produced from the recombinant Nipah henipavirus genome; and
a Nipah henipavirus envelope comprising F, G and M proteins of Nipah virus,
wherein the VRP can infect human cells, but cannot produce Nipah henipavirus particles from the infected human cells.

2. The Nipah henipavirus viral replicon particle of claim 1, wherein the deletion in the nucleic acid sequence encoding the F protein is a deletion of the entire F gene.

3. The Nipah henipavirus viral replicon particle of claim 1, wherein the genome is from Nipah strain Malaysia and comprises a deletion of nucleotide 6366 to nucleotide 8707 of SEQ ID NO: 4.

4. The Nipah henipavirus viral replicon particle of claim 3, wherein the M protein comprises: a) an amino acid sequence at least 95% identical to SEQ ID NO: 3; or b) the amino acid sequence of SEQ ID NO:.

5. (canceled)

6. The Nipah henipavirus viral replicon particle of claim 1, wherein the G protein comprises a) an amino acid sequence at least 95% identical to SEQ ID NO: 2; or b) the amino acid sequence of SEQ ID NO: 2.

7. (canceled)

8. The Nipah henipavirus viral replicon particle of claim 1, wherein the F protein comprises a) an amino acid sequence at least 95% identical to SEQ ID NO: 1; or b) the amino acid sequence of SEQ ID NO: 1.

9. (canceled)

10. An immunogenic composition comprising an effective amount of the Nipah henipavirus viral replicon particle of claim 1 and a pharmaceutically acceptable carrier.

11. The immunogenic composition of claim 10, further comprising an adjuvant.

12. A method of producing Nipah henipavirus virus replicon particles, comprising:
expressing Nipah henipavirus F protein from a host cell that is stably transfected with a nucleic acid molecule encoding the Nipah henipavirus virus F protein operably linked to a promoter; and
contacting the host cell with the viral replicon particle of claim 1, and
collecting Nipah henipavirus virus replicon particles produced by the host cell.

13. The method of claim 12, wherein the promoter is a constitutive promoter.

14. The method of claim 12, wherein the host cell is a human host cell.

15. The method of claim 12, wherein the nucleic acid molecule encoding the Nipah henipavirus virus F protein is codon optimized for expression in the host cell.

16. The method of claim 15, wherein the nucleic acid molecule encoding the Nipah henipavirus virus F protein comprises the nucleic acid sequence of SEQ ID NO: 5.

17. A method of inducing an immune response to Nipah henipavirus in a subject, comprising administering to the subject an effective amount of the immunogenic composition of claim 11, thereby inducing the immune response to the Nipah henipavirus.

18. The method of claim 17, wherein the composition is administered intranasally, intravenously, intramuscularly or subcutaneously.to the subject.

19. The method of claim 18, wherein the composition is administered intranasally to the subject.

20. The method of claim 17, wherein the subject is a human.

21. (canceled)

22. The method of claim 17, wherein only one dose of the immunogenic composition is administered to the subject.

23. The method of claim 17, wherein the method comprises a prime-boost immunization.

24. The method of claim 17, wherein the subject is infected with a Nipah henipavirus, and the method reduces at least one clinical symptom of the Nipah henipavirus infection.

25. (canceled)

26. The method of claim 17, wherein the subject is at risk of exposure to a Nipah henipavirus.

27. The method of claim 17, wherein the method induces the production of antibodies to the Nipah henipavirus in the subject.


SEQUENCE LISTING (6 sequences)

SEQ ID NO: 1 - protein, 546 aa, Nipah henipavirus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 2 - protein, 602 aa, Nipah henipavirus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 3 - protein, 352 aa, Nipah henipavirus
MEPDIKSISSESMEGVSDFSPSSWEHGGYLDKVEPEIDENGSMIPKYKIYTPGANERKYNNYMYLICYGFVEDVERTPETGKRKKIRTIAAYPLGVGKSASHPQDLLEELCSLKVTVRRTAGSTEKIVFGSSGPLNHLVPWKKVLTSGSIFNAVKVCRNVDQIQLDKHQALRIFFLSITKLNDSGIYMIPRTMLEFRRNNAIAFNLLVYLKIDADLSKMGIQGSLDKDGFKVASFMLHLGNFVRRAGKYYSVDYCRRKIDRMKLQFSLGSIGGLSLHIKINGVISKRLFAQMGFQKNLCFSLMDINPWLNRLTWNNSCEISRVAAVLQPSIPREFMIYDDVFIDNTGRILKG

SEQ ID NO: 4 - DNA, 18246 bp, Nipah henipavirus
  source 1..18246: mol_type = genomic DNA
accaaacaagggagaatatggatacgttaaaatatataacgtatttttaaaacttaggaaccaagacaaacacttttggtcttggtattggatcctcaagaaatatatcatcatgagtgatatctttgaagaggcggctagttttaggagttatcaatctaagttagggagagatgggagggctagtgcagcaactgctactttgacaaccaagataaggatatttgtaccagctactaatagtccagagctcagatgggaactaacattgtttgcacttgatgtgattagatctccgagtgctgccgagtcaatgaaagttggagctgctttcacactcatctctatgtattcagagagacccggggctctcattagaagtctcctcaatgacccagacattgaagctgtaataatagatgttggatcaatggtcaacggaataccagtaatggagaggagaggagacaaggctcaggaggagatggaaggcttgatgagaatcctcaaaactgctcgagacagcagcaagggaaaaacaccttttgttgacagccgagcttacggcctacggataacagacatgagcaccctggtctctgcagttatcaccatcgaggcccagatctggatactgatcgctaaagcagttacagctcccgacactgccgaggaaagtgaaactagaagatgggctaaatacgtccaacaaaagagagtcaatccgttctttgctctaactcagcaatggctaacagaaatgaggaatctgctctcccagagtctatcagtaaggaagttcatggttgagatcctcatagaagtcaagaaaggaggatctgctaaaggcagagcagtagaaataatctcagacatcggaaactatgtcgaggaaactggtatggcaggattcttcgcaaccatcagattcgggttggagacaaggtatccagcacttgcactcaacgaattccagagtgacctcaacaccatcaaaagcttgatgctactctacagagaaattggcccaagagccccttatatggtgcttcttgaagaatcaattcagactaaatttgcccctggaggttacccattattgtggagctttgccatgggtgtggctactactattgacaggtctatgggggcattgaatatcaatcgtggttatcttgagcctatgtatttcagactaggccaaaaatcagcacgtcaccatgctggaggaattgatcagaacatggcaaatagactgggactaagttcagatcaagttgcagaactcgctgctgcagttcaggaaacatcagcaggaaggcaagagagtaatgttcaggctagagaggcaaaatttgctgcaggaggtgtgctcattggaggcagtgatcaagatatcgatgaaggggaagaacctatagaacagagtggcagacagtcagttaccttcaaaagggagatgagtatttcatcccttgctaacagtgtgccgagcagttctgtgagcacatccggtgggaccagattgactaattcattactaaacctcagatcaagactggctgcaaaagcagcaaaagaagccgcctcatccaatgcaacagatgatccagcaatcagcaacagaactcaaggggaatcagagaagaagaataatcaagacctcaaacctgctcaaaatgaccttgatttcgtcagagctgatgtgtgacgtctatttccaatattctacagtatccaaaaatctttctatagtacactatcataatacgacactaagggatcaaccatatcaaagttacgaatcgttttaattatattaatcaaatgatactcttttatgggcaaaccgaagaaccaatgtctacatgtaaattgagctttggtattgcaatctaatacttgctcaaaatcttgaactattagtgtaatttctatcatcatagagttatcaagattttattatataagttggtgcagatctttggacatgaattacacactacactctaatgaagacaaaatttacattacatatttaaggactatttcctatcctttcaatggtacttggttatgaaggtttcttaatttaactaagctactgtctttgcactggaatatacaatacctcttacctcatttcttactttaatatcatgttatttttttgataagtcacttaacttgaccaaggtctaccaggtaatgctcgcacaagtgaactgcaatctcaacttagattaaacataatcatgcaaaatcactattttgtactactaactcattaagaaaaacttaggatccaagagatttactctaggatctcctattaagcttagcagtcattagttgagagttcaacttgcaaaactctaaccttcactctaataacaattcatccaatggataaattggaactagtcaatgatggcctcaatattattgactttattcagaagaaccaaaaagaaatacagaagacatacggacgatcaagtattcaacaacccagcatcaaagatcaaacaaaagcctgggaagattttctgcagtgcaccagtggagaatctgaacaagttgaggggggaatgtctaaggatgatggagatgttgaaagaagaaacttggaggatctatccagtacttctcccacagatggaactattggaaagagagtgtcgaacacccgtgactgggcagaaggttcagatgacatacaactggacccagtggttacagacgttgtataccatgatcatggaggagaatgtaccggatatggatttacttcaagccctgagagagggtggagtgattacacatcaggagcaaacaatgggaatgtatgtcttgtatctgatgcaaagatgctgtcctatgctcccgaaattgcagtttctaaagaagatcgggaaactgatctagttcatcttgagaataaactatctactacaggactgaatcccacagcagtaccgttcactctgagaaacctgtctgatcctgcaaaagactctcctgtgattgctgaacactactacggactaggagttaaagagcaaaacgttggccctcagactagcagaaatgtcaatttggacagcatcaaattgtacacatcagatgacgaagaggcagatcagcttgaattcgaagatgagtttgcaggaagctcaagtgaagtgatagtcggcatttctcctgaagatgaagagccttcaagtgttggcggaaaacccaatgaatccattggacgtacaatcgaaggccaatcaatccgagacaaccttcaagccaaggacaacaaatcaacagatgtaccaggagcaggaccgaaagattcagcagtgaaggaagaaccaccccagaagaggctacctatgttagctgaagaatttgagtgctctggatcggaagacccaatcattcgggagctgctgaaggagaactcactcataaattgtcagcaagggaaagatgctcagcctccatatcattggagcatcgagaggtcaataagcccggataaaactgagatcgtcaacggtgctgtgcaaactgctgacaggcaaagaccaggaactccgatgccaaagtcccgaggtattcccattaaaaagggcacagacgcgaaatatccatctgctgggacggaaaacgtgcctgggtcgaagagtggtgcaacccggcatgttcgaggatcacccccctaccaagaaggcaagagtgtcaatgcggagaatgtccaactgaatgcttccactgcggttaaggaaactgataagtcagaagtaaaccccgtagacgacaacgactcacttgatgataaatacatcatgccttcagatgatttctcaaacactttcttcccgcacgacactgatcgcttgaattatcacgcagatcatttaggtgattatgaccttgaaaccctgtgtgaagagtcggttctaatgggagtgatcaactctataaaattaattaatctggatatgcgcttaaatcacattgaagaacaagttaaagagatcccaaagatcatcaataagcttgagtccattgacagagttctggccaagactaacaccgcactctcaaccattgaaggacacctggtttccatgatgataatgataccagggaaagggaaaggagaaagaaaggggaaaaataatcctgagcttaaaccagtgataggaagagacattctagagcagcaatctcttttttcttttgacaatgtcaagaatttcagagatggatcgttgacaaacgaaccgtatggggcagctgtacagttgagagaagatcttattcttcctgaacttaattttgaggagacaaatgcatctcaatttgttcctatggcagatgattcatccagagatgttatcaagacattgataaggactcacattaaagatagagagttgagatcagaactgattggttacctgaataaagcggaaaatgatgaggaaattcaggagatagcgaacactgtcaatgacatcattgacggtaatatttgatcactgaattgtcagcagaaatacaatgatctaacaacaatctcccacaagtagacaatggtttcaggtcaataataacaacctcaatactaatctttcacataagcattactcattccagccctcagacgataacacaatacttgatacatgtttattgaagtgtatgtagcatgattgaactattcaataactgtatttctcactcttgctcttagttagtcattgtgtctaataattattattacagtacaaggtattatgaattcaaagatacgcaataaatctgatatcagcatagagtagaaaattgttgtttttgtcatgatcattcgaagatttaacaatgatgtcaactttcatacctaaacataataacataaaatggtcgatttgtattgtagatctctcacgcattttagtgtcatgaattagtgtttcaaatcagttgcatatcaattaagaaaaacttaggagacaggtatagaacctctctttcagataactggtcaattaaggacagaaattctgtttctcaaatccgctagcctttgtcaaagaggacacaagcaatggagccggacatcaagagtatttcaagtgagtcaatggaaggagtatctgatttcagccctagttcttgggagcatggtgggtatcttgataaggttgaaccagaaattgatgaaaatggcagtatgattccaaaatacaagatctataccccaggagctaacgagaggaaatacaacaactacatgtaccttatatgttacggctttgttgaagatgttgagagaaccccagagacagggaaacgcaagaagatcaggacaattgctgcctaccctctgggtgttggtaagagtgcctctcatccccaagatcttctggaggaactctgttccctcaaagttactgtgagaagaacagctggatcaactgagaaaattgtgtttggatcatctggccctctaaatcacctcgttccgtggaagaaagtactgactagtggttcaatttttaatgcagtcaaggtttgtcggaacgttgatcagatacagcttgacaagcatcaagctctgagaatattttttctcagtatcacaaagctcaatgattctggaatctacatgattccacgaaccatgcttgagttcaggagaaacaatgccattgccttcaatcttctagtgtacttgaagattgatgctgatttatccaaaatggggatccagggaagcctcgataaagatggcttcaaggttgcctccttcatgctacacttggggaactttgtccgtcgtgcagggaagtattactctgttgattattgtaggaggaagattgataggatgaaattgcagttttcactgggttccataggcggactaagtctccacattaagatcaatggtgtaatcagcaaacggctgtttgctcaaatgggattccaaaaaaacctttgtttctctttgatggacatcaatccttggctcaacagattgacctggaacaacagttgtgagatcagccgagtagcagctgtgttgcagccttctattccaagagagttcatgatctatgatgatgtcttcattgacaatacagggagaattctaaagggctaaacagaattcttctaaaatttaatcagtcatgagtttagtaatcatacctagtcataatacatcacacaggactatttacaaaagacagttaaaaaatggaataatcatgtagtagtaattgagaacattattagaatagtataactaaaatgtagtttttttgagtatttgatttaaaattagataactattacaaaaaacttaggagccaagctcttgcctcgttcagaaggttaaacaagcattcttaccattggatcaacaaaaggattggttttatcgtctaagaaatttattgaaaggcaaagaaattcctggttttatgttgaatgaggtgtatcaaactaaggagaccttctaacagccaggtcataggaatataaataaaaataagaataaaattgattccatcggaagattcatttcaagaagtgatcaaatcaaagcggttggcagacctaccaatcatataccacaagactcgacaatggtagttatacttgacaagagatgttattgtaatcttttaatattgattttgatgatctcggagtgtagtgttgggattctacattatgagaaattgagtaaaattggacttgtcaaaggagtaacaagaaaatacaagattaaaagcaatcctctcacaaaagacattgttataaaaatgattccgaatgtgtcgaacatgtctcagtgcacagggagtgtcatggaaaattataaaacacgattaaacggtatcttaacacctataaagggagcgttagagatctacaaaaacaacactcatgaccttgtcggtgatgtgagattagccggagttataatggcaggagttgctattgggattgcaaccgcagctcaaatcactgcaggtgtagcactatatgaggcaatgaagaatgctgacaacatcaacaaactcaaaagcagcattgaatcaactaatgaagctgtcgttaaacttcaagagactgcagaaaagacagtctatgtgctgactgctctacaggattacattaatactaatttagtaccgacaattgacaagataagctgcaaacagacagaactctcactagatctggcattatcaaagtacctctctgatttgctttttgtatttggccccaaccttcaagacccagtttctaattcaatgactatacaggctatatctcaggcattcggtggaaattatgaaacactgctaagaacattgggttacgctacagaagactttgatgatcttctagaaagtgacagcataacaggtcaaatcatctatgttgatctaagtagctactatataattgtcagggtttattttcctattctgactgaaattcaacaggcctatatccaagagttgttaccagtgagcttcaacaatgataattcagaatggatcagtattgtcccaaatttcatattggtaaggaatacattaatatcaaatatagagattggattttgcctaattacaaagaggagcgtgatctgcaaccaagattatgccacacctatgaccaacaacatgagagaatgtttaacgggatcgactgagaagtgtcctcgagagctggttgtttcatcacatgttcccagatttgcactatctaacggggttctgtttgccaattgcataagtgttacatgtcagtgtcaaacaacaggcagggcaatctcacaatcaggagaacaaactctgctgatgattgacaacaccacctgtcctacagccgtactcggtaatgtgattatcagcttagggaaatatctggggtcagtaaattataattctgaaggcattgctatcggtcctccagtctttacagataaagttgatatatcaagtcagatatccagcatgaatcagtccttacaacagtctaaggactatatcaaagaggctcaacgactccttgatactgttaatccatcattaataagcatgttgtctatgatcatactgtatgtattatcgatcgcatcgttgtgtatagggttgattacatttatcagttttatcattgttgagaaaaagagaaacacctacagcagattagaggataggagagtcagacctacaagcagtggggatctctactacattgggacatagtgtattcagattgatgaaattatgttagagaaatcagaaaacttctgactttcagaaatggattgtatacaattagttagatcatcctgaataatcgaggtgagaacattgcaactataaaatcagatcatgtaaatagttgtaaaaaattaaaagcttcttttaattcttttgaacaataatttaattaatatataacatattctctcacacgagcgctaacctatacactctctactaatattttatactcataattaatgatataatgacaaataaggattcaaattggattatgatatagtttcatactacaatagcatttcgaccaagaaaatatccttacaattatacaatgtacttaaccgtgaatatgtaattgataatttccctttagaaatttaataaaaaacttaggacccaggtccataactcattggatacttaactgtatctttctaagctatcacatatcaaaggagagattgaatgcttttttggagatctagatcattactatatgtgtctcctataatcacatcataggagtgaaccataatacacatctttgggtaggggaaggaaagtattgttgacgtactgattgatctgcttgagtcaaataatcagtcataacaattcaagaaaatgccggcagaaaacaagaaagttagattcgaaaatactacttcagacaaagggaaaattcctagtaaagttattaagagctactacggaaccatggacattaagaaaataaatgaaggattattggacagcaaaatattaagtgctttcaacacagtaatagcattgcttggatctatcgtgatcatagtgatgaatataatgatcatccaaaattacacaagatcaacagacaatcaggccgtgatcaaagatgcgttgcagggtatccaacagcagatcaaagggcttgctgacaaaatcggcacagagatagggcccaaagtatcactgattgacacatccagtaccattactatcccagctaacattgggctgttaggttcaaagatcagccagtcgactgcaagtataaatgagaatgtgaatgaaaaatgcaaattcacactgcctcccttgaaaatccacgaatgtaacatttcttgtcctaacccactcccttttagagagtataggccacagacagaaggggtgagcaatctagtaggattacctaataatatttgcctgcaaaagacatctaatcagatattgaagccaaagctgatttcatacactttacccgtagtcggtcaaagtggtacctgtatcacagacccattgctggctatggacgagggctattttgcatatagccacctggaaagaatcggatcatgttcaagaggggtctccaaacaaagaataataggagttggagaggtactagacagaggtgatgaagttccttctttatttatgaccaatgtctggaccccaccaaatccaaacaccgtttaccactgtagtgctgtatacaacaatgaattctattatgtactttgtgcagtgtcaactgttggagaccctattctgaatagcacctactggtccggatctctaatgatgacccgtctagctgtgaaacccaagagtaatggtgggggttacaatcaacatcaacttgccctacgaagtatcgagaaagggaggtatgataaagttatgccgtatggaccttcaggcatcaaacagggtgacaccctgtattttcctgctgtaggatttttggtcaggacagagtttaaatacaatgattcaaattgtcccatcacgaagtgtcaatacagtaaacctgaaaattgcaggctatctatggggattagaccaaacagccattatatccttcgatctggactattaaaatacaatctatcagatggggagaaccccaaagttgtattcattgaaatatctgatcaaagattatctattggatctcctagcaaaatctatgattctttgggtcaacctgttttctaccaagcgtcattttcatgggatactatgattaaatttggagatgttctaacagtcaaccctctggttgtcaattggcgtaataacacggtaatatcaagacccgggcaatcacaatgccctagattcaatacatgtccagagatctgctgggaaggagtttataatgatgcattcctaattgacagaatcaattggataagcgcgggtgtattccttgacagcaatcagaccgcagaaaatcctgtttttactgtattcaaagataatgaaatactttatagggcacaactggcttctgaggacaccaatgcacaaaaaacaataactaattgttttctcttgaagaataagatttggtgcatatcattggttgagatatatgacacaggagacaatgtcataagacccaaactattcgcggttaagataccagagcaatgtacataaaaatcaacctcataatttaatggattgatctaatataatgataataatcgtacaaagacatgtgatgtaaacaaaattgttgtaattaaataagtcctcagctgaatacttttttaagattagcaatagcatgtttttccagttattggatagttgataatataattctgaaactgggttaataaataatcttgatcggtgatctttgagaacaatgatatcatatagttcatcaagtgataatcaattctttatatgtacactttagagtatattttgagacttagtattttcggcccgaatgttaaatttaatagttcatacataacctaaactcaagttctaagcataatgataacaattaatgcgaacttgtcttgatgtaaggaagatttgatattaactgagactccacttgatatagtagagctgaatcttgtaaataaattataatgaatagtttattcaaagattatcattcatattagtgtaaattaagaaaaacttaggacccaggtccttgattatgccaattttctcgagaaatcattcaattgaccatagactgaaagcgttgttacctagttcttcagaagagatcttattagaattaatttatatgatctaattcccttaaaaactgaataccaaaaaacaaaaatggccgatgaattatcaatatccgacatcatttaccctgaatgtcatttggatagtcctatagtctctggtaaactaatatcagctattgaatatgctcaattgagacacaatcagcccagtgatgataaaagactgtctgagaatattaggttaaaccttcacgggaaaagaaagagtctatacatattaagacaatccaaacagggtgattacattagaaacaacataaaaaacctaaaggaattcatgcatattgcgtaccctgaatgcaataacattctattctccatcacatcccaaggcatgactagcaaacttgataacatcatgaaaaagtcattcaaagcatacaatatcattagtaagaaagtaattgggatgctgcaaaatatcactagaaatctcataactcaagatagaagagatgaaataattaatatacatgagtgtaggcgattaggggatttagggaagaatatgagtcaatctaaatggtatgagtgttttttgttttggtttactatcaaaacagagatgcgagcagtgatcaagaattcgcaaaagccgaaattccgttcagattcatgcataatacacatgcgagacaaaagtactgaaataatcctaaatccgaatcttatctgcattttcaaatcagacaaaactggaaagaagtgttattatcttacacccgaaatggttctaatgtattgtgatgtcctagagggaaggatgatgatggagacaacagtcaaatcggatatcaagtaccaacctctaatctcgagatccaatgccctctgggggctaattgatcccttgttccctgtcatgggaaacagaatttacaatatagtgtctatgatagagcctttagttcttgcactactccaactcaaggatgaggctaggatcctgaggggtgcatttctgcatcactgcataaaggaaatgcatcaagaattgagtgagtgtggttttacagatcagaagattcggtctatgtttattgatgatcttttatccattctaaatatcgataatatacatctgttggcagagttcttttctttctttcgtacgtttggccatcctattcttgaggctaaagttgctgcagaaaaagtgagagaacatatgttggcagataaagttcttgaatatgcccctataatgaaagcacatgctatattctgcgggactataataaatgggtatagggatagacacggaggagcctggcctcctctttacctccccgcacatgcatctaaacatataatccgtttgaaaaattctggggaatctttgaccattgatgactgtgtcaagaattgggaatcattctgtgggattcaatttgattgtttcatggagctgaaattggacagtgatctgagtatgtatatgaaagataaagctttatctccaatcaaagacgaatgggacagtgtatacccacgtgaagtgttgagctataccccaccgaagtcaaccgagccaagaagattggttgacgtttttgtaaatgatgaaaactttgatccatacaacatgctggaatatgtcttatccggtgcttatctcgaggatgaacaattcaatgtttcttatagcttgaaggagaaagagacgaagcaagctggacgattgttcgcaaagatgacctacaaaatgcgtgcatgtcaagtcatagcagaggccctgatagcctcaggtgtcggtaaatattttaaggagaacgggatggttaaggatgagcacgaacttttgaagacactcttccaattgtctatttcctcagttcctcgagggaacagtcagggtaatgatcctcaatccatcaataatatagaaagagatttccaatactttaaaggggtcactaccaatgtgaaagacaaaaagaataactcttttaataaggttaaatctgctctcaataatccgtgccaagctgacggagtccatcataacatgtcacccaatacacgaaatcgttataagtgtagtaatacaagtaagtcttttctcgattatcataccgagtttaatcctcacaatcactataaatcagacaatacagaggcggccgtactgtccaggtatgaggacaacactgggacaaaatttgatacagtaagtgcatttcttacaactgatcttaagaaattctgtctcaattggagatacgaatcaatggctatatttgctgaacgtctggatgagatatacggtttacctggattttttaattggatgcacaaacgactagaaagatctgttatctatgttgcagaccctaattgcccccctaatattgacaaacatatggaactagaaaaaactcctgaagatgatatattcattcattatcctaaaggcggtattgaaggatatagccaaaaaacatggactatagcaactatcccctttttattcttgagtgcctatgagacaaacacgaggattgctgcaattgtccaaggagacaatgaatcaattgctatcactcaaaaagttcatcctaatcttccctacaaggtaaagaaagagatctgtgcaaagcaagctcagctttattttgaaaggttaaggatgaacttaagagccctcggccacaatcttaaagctacagaaactatcatcagtacacatctttttatttattcgaagaaaattcattatgatggtgctgtgctgtctcaggcactcaaatcaatgtcaagatgttgcttttggtcagagactctggtggatgaaactagatcagcttgtagtaacatcagcactacaatagctaaagctatagaaaatgggttgtcaagaaatgtcggctattgcatcaatattttgaaagtaattcagcagcttctcatatcaactgagtttagtattaacgagacattgacactggatgtgacatctcccatttcaaataatttagattggcttataacagctgcattaatcccggcacctattggaggattcaattaccttaatttgtctagaatttttgttagaaatataggtgatccggttacagcatctttggctgatcttaagagaatgattgatcacagtattatgactgaaagcgtattacaaaaagttatgaatcaagaacctggtgatgcgagtttcttggactgggccagtgatccatactcgggcaacttgcctgactcacaaagcatcactaaaacaattaaaaatatcacagcaaggactatactgaggaactcaccgaacccaatgctaaaaggtttatttcatgacaaatcttttgatgaagatcttgaactagctagcttcttaatggacaggagggttatattacctagagccgctcatgagatactggataattcattgacaggtgccagagaggaaattgctggtttattagatacaactaaaggcttgatcagatcagggctaagaaagagtggacttcagccaaagttagtttctagattatctcatcatgattataatcaatttttaatactgaacaaacttctatcaaacagaagacaaaatgacttgatatcatcaaatacttgctcagttgacttggcacgagcattgagatctcacatgtggagggaattagcgttaggtagagtaatatacggtcttgaggtaccagatgcacttgaggctatggtgggaaggtatataacagggagcttagagtgccaaatttgtgagcagggaaacacgatgtatgggtggttctttgtacctagggattcccaattggatcaggtagatagagagcactcatcaataagagtaccttatgtaggatcaagtacggatgaaagatcggatatcaaactagggaatgtcaaaagaccaactaaggccttgcgttctgctatcagaattgcgacagtatatacttgggcctatggggacaatgaagagtgttggtatgaagcttggtacctagcgtctcagagggtaaacatagacttagatgtattgaaagctataaccccagtttccacttcaaacaatttatcccatagattgagagataaatccacacaatttaagtttgcagggagtgtactcaacagagtttctagatatgttaacataagcaatgacaatctagatttcagaattgagggagaaaaggtagatacgaatcttatttatcaacaagcaatgctattagggttatcggtattggaaggtaaattcagattgagattagaaactgatgattacaacgggatatatcacttacacgtaaaggataattgttgtgtcaaagaagtggctgatgtaggccaagtagacgctgagttgcctatcccagaatatactgaagtggataacaatcatcttatatatgatccagaccccgtttcagaaatagattgcagccgtctttctaatcaggagtccaaatcaagagaattagactttcctttatggtcaactgaggaacttcatgatgtcctagctaagactgttgctcagaccgttcttgagattataacaaaggctgacaaggatgttttaaagcaacaccttgcaatagactctgacgataacatcaacagcttaatcacagaatttctaatagttgatcctgaactgtttgcactttatctaggacaatctatatcaataaaatgggcctttgaaattcatcataggcgtcctagaggaagacatactatggtcgacctattgtcagatcttgtatcaaatacatcaaagcacacttacaaagtgttgtcaaatgccttgtcacatcctagagtattcaagagatttgtaaactgtggcttgctattgcctacacagggtccttaccttcatcaacaagattttgaaaagttgtctcaaaaccttcttgtaacatcttatatgatttatctaatgaactggtgtgacttcaagaaatccccctttttaatcgccgaacaggatgaaactgtgataagtctacgagaggatataataacatccaaacatctctgtgttataattgacttatatgcaaatcaccataaacctccttggataatagatctaaacccacaagaaaaaatatgtgtactgcgtgactttatttctaaatctaggcatgtggacacgtcctccagatcatggaatacttctgacctggattttgtaatattctatgcatctttgacttatttgagaagaggtataataaaacaattaaggataagacaagttactgaggttatagataccacaacaatgttaagggacaatataattgtagagaatcctcctattaaaacaggagtgttagacatcagaggttgtataatatacaatttagaggaaatcctgtctatgaacacaaaatcagcatcaaaaaagatctttaatcttaatagtaggccgtcagtggagaatcataaatatagaaggataggtctcaactcatcatcttgttacaaggcattaaatctatcacctctgattcaaaggtatttgccgtcgggagctcaaaggttgtttataggagaaggttctgggagcatgatgttattatatcagtctacattggggcaatcaatttctttttacaattcaggtatagatggagattatataccaggtcaaagagaactgaaactatttccctctgaatactcaattgctgaggaagacccatctctgacggggaaattgaaaggactagtggtgcccctattcaatggaagaccagaaacaacatggatcgggaatttagactcctacgagtatatcataaataggacagcggggcgaagtataggtcttgtccattctgacatggagtctgggattgacaaaaatgtagaggagatactagtagaacattcccatctaatatctatcgcgataaatgttatgatggaggacggactattagtatccaagatagcatacacccctggattcccaatctcaagattatttaacatgtacagatcatatttcggactagtactggtgtgtttcccagtatatagtaatccagattctactgaagtatatcttctttgcttacagaagacggtcaagactattgttcccccgcaaaaagtccttgagcactctaatttgcacgatgaagtcaatgaccagggaataacatcagtgatttttaaaatcaagaattcacagtctaagcagttccacgatgatctaaagaagtactatcagattgaccaacctttttttgtaccaactaaaatcactagtgatgaacaagtacttctccaagcagggctgaaactcaatgggccagaaattcttaagagtgaaatcagttatgatatcggttcagatatcaatacattaagagacaccatcataattatgttaaatgaggctatgaattattttgatgacaacagatcaccttcacaccacctagaaccctatccagttttggagagaactagaattaaaacaataatgaattgtgtgactaaaaaagtgattgtctactcacttatcaagttcaaggacaccaaaagctcagaactttatcacatcaaaaataacatcagaagaaaagttctaatcttagatttcagatcgaagctcatgacaaagactctacctaaagggatgcaagagagaagagaaaaaaacggtttcaaagaagtttggatagtagatttatcgaatcgagaagttaaaatctggtggaagataatcggatacatatctattatctgatttaaccttccaaatccaagaccaactgataacttatgttgatctaaggttcagttattaagaaaaacttaataacgattcttctttacccttgttcggt

SEQ ID NO: 5 - DNA, 1641 bp, synthetic construct
  source 1..1641: mol_type = other DNA
atggtggtcattctggacaaacggtgttactgcaacctgctgattctgattctgatgattagcgagtgtagtgtgggcattctgcactacgagaaactgtccaagattggactggtgaaaggcgtcactcggaagtataaaatcaagtctaaccccctgaccaaagacatcgtgattaagatgatccctaacgtcagtaatatgtcacagtgcacagggtccgtgatggagaactacaagaccagactgaatggaattctgacacccatcaaaggcgccctggaaatctataagaacaatactcacgacctggtgggggatgtcaggctggcaggagtgattatggcaggggtcgccatcggaattgccactgccgctcagatcaccgctggagtggcactgtacgaggccatgaagaacgctgacaacattaacaagctgaagagcagcatcgagtctaccaatgaagctgtggtcaaactgcaggagacagcagaaaagactgtgtacgtcctgacagcactgcaggactatatcaacacaaatctggtgcctactatcgataaaattagttgtaagcagaccgaactgtcactggacctggctctgagcaagtacctgtccgatctgctgttcgtgtttggcccaaacctgcaggatcccgtctctaatagtatgacaatccaggcaattagccaggccttcggcgggaactacgagaccctgctgcgcacactgggctatgccactgaggactttgacgatctgctggaatcagatagcatcaccgggcagatcatctacgtggacctgtctagttactatatcattgtgcgagtctacttcccaattctgaccgagatccagcaggcctatatccaggaactgctgcccgtgagcttcaacaatgataactctgagtggatcagtattgtgcctaattttattctggtccgcaacacactgatcagcaatatcgaaattggcttttgcctgattactaaacgaagcgtgatctgtaatcaggactacgccacccctatgacaaacaatatgcgggagtgcctgactggaagcaccgagaagtgtccccgggagctggtggtctcaagccatgtgccacggttcgccctgagcaacggcgtgctgtttgctaattgcatctccgtcacctgccagtgtcagaccacagggcgcgctatttcccagtctggagagcagacactgctgatgatcgataacactacctgtccaactgcagtgctgggcaatgtcatcattagcctgggcaaatacctggggtccgtgaactataattctgaaggaatcgccattggcccccctgtgttcaccgacaaggtcgatatttcctctcagatcagttcaatgaaccagtcactgcagcagagcaaagactacatcaaggaggcccagagactgctggatactgtgaatccaagcctgatttccatgctgtctatgatcattctgtatgtgctgagtatcgcttcactgtgcatcgggctgattaccttcatctcttttatcattgtggagaagaaaaggaacacctacagtcgcctggaagatcggagagtgagacccacaagctccggcgacctgtactatattgggacatga

SEQ ID NO: 6 - protein, 604 aa, Hendra henipavirus
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES